(19) United States
(12) Patent Application Publication
(10) Pub. No.: US 2026/0028424 A1
(43) Pub. Date: Jan. 29, 2026

(54) BISPECIFIC ANTI-C-KIT AND ANTI-CD203C ANTIGEN-BINDING MOLECULES AND USES THEREOF

(71) Applicant: Granular Therapeutics Limited, Sandwich (GB)

(72) Inventors: William James Jonathan FINLAY, Glasgow (GB); Orla Margaret CUNNINGHAM, Coatbridge (GB)

(21) Appl. No.: 18/998,577

(22) PCT Filed: Jul. 27, 2023
(86) PCT No.: PCT/EP2023/070937

§ 371 Date: Jan. 27, 2025

Related U.S. Application Data
(60) Provisional application No. 63/393,036, filed on Jul. 28, 2022.

(51) Int. Cl. C07K 16/40; A61K 47/68; A61P 35/02; C12N 15/11; C12N 15/63

(52) U.S. Cl. CPC C07K 16/40; A61K 47/6871; A61P 35/02; C12N 15/11; C12N 15/63; C07K 2317/31; C07K 2317/51; C07K 2317/515; C07K 2317/565

(57) ABSTRACT

Provided herein are bispecific antigen-binding molecules that bind to c-Kit and CD203c. Further provided herein are therapeutic uses of and methods of producing such molecules and related pharmaceutical compositions, nucleic acid molecules, expression vectors and host cells.

### CROSS-REFERENCE TO RELATED APPLICATIONS

This application claims the benefit of U.S. Provisional Patent Application No. 63/393,036, filed on Jul. 28, 2022, the disclosure of which is hereby incorporated by reference in its entirety.

### REFERENCE TO AN ELECTRONIC SEQUENCE LISTING

The contents of the electronic sequence listing (UHFL_002_01WO_SeqList_ST26.xml; Size:71,462 bytes; and Date of Creation: Jul. 24, 2023) are herein incorporated by reference in their entirety.

### TECHNICAL FIELD

The disclosure is related to bispecific antigen-binding molecules and therapeutic uses of such molecules.

### BACKGROUND

Granulocytes (e.g., mast cells and basophils) are central drivers of pathology in many chronic inflammatory diseases. Existing approaches for treating such diseases target the mast cell survival receptor c-Kit. While this approach has been clinically validated to effectively deplete mast cells and ameliorate associated inflammation, indiscriminate inhibition of c-Kit can lead to on-target dose-limiting bone marrow suppression. These adverse events are difficult to avoid with high affinity target engagement driven by bivalent engagement with standard antibodies. There is a paucity of granulocyte cell-selective drugs, which leaves a significant unmet medical need for patients suffering from mast cell driven disease.

### SUMMARY

Provided herein are bispecific proteins comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c.

Provided herein is an anti-c-Kit and anti-CD203c bispecific protein, wherein The bispecific protein of claim 1, wherein the anti-c-Kit VH domain comprises a heavy chain complementarity determining region 1 (HCDR1) comprising the amino acid sequence of SEQ ID NO: 2, a heavy chain complementarity determining region 2 (HCDR2) comprising the amino acid sequence of SEQ ID NO: 3, and a heavy chain complementarity determining region 3 (HCDR3) comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 73; and the VL domain comprises a light chain complementarity determining region 1 (LCDR1) comprising the amino acid sequence of SEQ ID NO: 6, a light chain complementarity determining region 2 (LCDR2) comprising the amino acid sequence of SEQ ID NO: 7, and a light chain complementarity determining region 3 (LCDR3) comprising the amino acid sequence of SEQ ID NO: 8.

Provided herein is an anti-c-Kit and anti-CD203c bispecific protein, wherein the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 10, a HCDR2 comprising the amino acid sequence of SEQ ID NO: 11, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

Provided herein is an anti-c-Kit and anti-CD203c bispecific protein, wherein the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

Provided herein is an anti-c-Kit and anti-CD203c bispecific protein, wherein the anti-c-Kit VH domain comprises the amino acid sequence of SEQ ID NO: 65; the anti-c-CD203c VH domain comprises the amino acid sequence of SEQ ID NO: 71; and the VL domain comprises the amino acid sequence of SEQ ID NO: 5.

Provided herein is an anti-c-Kit and anti-CD203c bispecific protein, wherein the anti-c-Kit VH domain comprises the amino acid sequence of SEQ ID NO: 1; the anti-c-CD203c VH domain comprises the amino acid sequence of SEQ ID NO: 9; and the VL domain comprises the amino acid sequence of SEQ ID NO: 5.

Provided herein is an anti-c-Kit and anti-CD203c bispecific protein, wherein the anti-c-Kit VH domain comprises the amino acid sequence of SEQ ID NO: 1; the anti-c-CD203c VH domain comprises the amino acid sequence of SEQ ID NO: 13; and the VL domain comprises the amino acid sequence of SEQ ID NO: 5.

Provided herein is an anti-c-Kit and anti-CD203c bispecific protein, wherein the anti-c-Kit VH domain comprises the amino acid sequence of SEQ ID NO: 1; the anti-c-CD203c VH domain comprises the amino acid sequence of any one of SEQ ID NOs: 16-51; and the VL domain comprises the amino acid sequence of SEQ ID NO: 5.

Provided herein is an anti-c-Kit and anti-CD203c bispecific protein, wherein the anti-c-Kit VH domain comprises the amino acid sequence of any one of SEQ ID NOs: 63-67; the anti-c-CD203c VH domain comprises the amino acid sequence of any one of SEQ ID NOS: 68-72; and the VL domain comprises the amino acid sequence of SEQ ID NO: 5.

In some embodiments, the bispecific protein comprises an immunoglobulin constant region. In some embodiments, the immunoglobulin constant region is an IgG, IgE, IgM, IgD, IgA or IgY immunoglobulin constant region. In some embodiments, the immunoglobulin constant region is an IgG1, IgG2, IgG3, IgG4, IgA1 or IgA2 immunoglobulin constant region.

In some embodiments, the immunoglobulin constant region is an immunologically inert constant region.

In some embodiments, the first polypeptide chain comprises a first immunoglobulin constant region and the second polypeptide chain comprises a second immunoglobulin constant region, and wherein the first immunoglobulin constant region and the second immunoglobulin constant region comprise knob-in-hole mutations.

In some embodiments, the first immunoglobulin constant region comprises a CH3 domain comprising the amino acid substitutions S354C and T366W, and wherein the second immunoglobulin constant region comprises a CH3 domain comprising the amino acid substitutions Y349C, T366S, L368A, and Y407V, wherein numbering is according to the EU index as in Kabat.

In some embodiments, the first immunoglobulin constant region comprises a CH3 domain comprising the amino acid substitutions Y349C, T366S, L368A, and Y407V, and wherein the second immunoglobulin constant region comprises a CH3 domain comprising the amino acid substitutions S354C and T366W, wherein numbering is according to the EU index as in Kabat.

Provided herein is a bispecific protein, wherein (a) the first immunoglobulin constant region comprises the amino acid sequence of SEQ ID NO: 52, and the second immunoglobulin constant region comprises the amino acid sequence of SEQ ID NO: 53; or (b) the first immunoglobulin constant region comprises the amino acid sequence of SEQ ID NO: 53, and the second immunoglobulin constant region comprises the amino acid sequence of SEQ ID NO: 52.

Provided herein are bispecific proteins that bind c-Kit and CD203c, wherein the c-Kit and CD203c are on the surface of the same cell.

In some embodiments, a bispecific protein is a designed ankyrin repeat protein (DARPin), a tandem VHH, or a tandem immunoglobulin new antigen receptor (IgNAR).

Provided herein is an immunoconjugate comprising a bispecific protein disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising a bispecific protein or an immunoconjugate disclosed herein, and a pharmaceutically acceptable carrier, diluent or excipient.

Provided herein is a nucleic acid molecule encoding a bispecific protein disclosed herein. Provided herein is a nucleic acid molecule encoding the first polypeptide chain, the second polypeptide chain, the third polypeptide chain, and the fourth polypeptide chain of a bispecific protein disclosed herein.

Provided herein is an expression vector comprising a nucleic acid molecule disclosed herein.

Provided herein is a recombinant host cell comprising a nucleic acid molecule or an expression vector disclosed herein.

Provided herein is a method of producing a bispecific protein, the method comprising: culturing a recombinant host cell disclosed herein under conditions whereby the nucleic acid molecule is expressed, thereby producing the protein; and isolating the protein from the host cell or culture.

Provided herein is a method for treating an inflammatory disease or a neoplasm in a subject in need thereof, the method comprising administering to the subject a therapeutically effective amount of a bispecific protein, an immunoconjugate, or a pharmaceutical composition disclosed herein.

Provided herein is a method for ameliorating a symptom of an inflammatory disease or a neoplasm in a subject in need thereof, the method comprising administering to the subject a therapeutically effective amount of a bispecific protein, an immunoconjugate, or a pharmaceutical composition disclosed herein.

In some embodiments of the methods provided herein, the inflammatory disease is a chronic inflammatory disease. In some embodiments of the methods provided herein, the inflammatory disease is a mast cell-driven disease. In some embodiments of the methods provided herein, the inflammatory disease is chronic urticaria, chronic pruritus, atopic dermatitis, allergic asthma, prurigo nodularis, eosinophilic gastritis, eosinophilic duodenitis, or eosinophilic esophagitis.

In some embodiments of the methods provided herein, the neoplasm is a mast cell-driven neoplasm. In some embodiments of the methods provided herein, the neoplasm is systemic mastocytosis or mast cell leukemia.

Provided herein is a bispecific protein, an immunoconjugate, or a pharmaceutical composition disclosed herein for use as a medicament.

Provided herein is a bispecific protein, an immunoconjugate, or a pharmaceutical composition disclosed herein for use in the treatment of an inflammatory disease or a neoplasm.

In some embodiments of the uses disclosed herein, the inflammatory disease is a chronic inflammatory disease. In some embodiments of the uses disclosed herein, the inflammatory disease is a mast cell-driven disease. In some embodiments of the uses disclosed herein, the inflammatory disease is chronic urticaria, chronic pruritus, atopic dermatitis, allergic asthma, prurigo nodularis, eosinophilic gastritis, eosinophilic duodenitis, or eosinophilic esophagitis.

In some embodiments of the uses disclosed herein, the neoplasm is a mast cell-driven neoplasm. In some embodiments of the uses disclosed herein, the neoplasm is systemic mastocytosis or mast cell leukemia.

### BRIEF DESCRIPTION OF THE DRAWINGS

FIG. 1 depicts a diagram showing that a bispecific format ensures specificity for activated mast cells. Monovalent anti-c-Kit has a low functional affinity for c-Kit on hematopoietic stems cells (HSCs). Without the stabilization of binding provided by co-engagement with CD203c, the monovalent binding is transient and results in low c-Kit inhibition potency that cannot effectively compete with high affinity binding of endogenous dimeric SCF ligand. However, on dual receptor expressing cells, bivalent binding can occur, SCF can no longer compete for receptor occupancy, and c-Kit signaling is ablated.

FIG. 2 depicts a schematic representation of Knob-in-hole bispecific format. To enable streamlined production and purification, a common light chain bispecific format was used for dual c-Kit/CD203c targeting. This is in combination with engineered cysteine residues Knob-in-hole mutations in the CH3 Fc domain that drive heterodimeric heavy chain pairing. Exemplary common light chain and heterodimeric heavy chain sequences are provided in Tables 1-3.

FIG. 3A-FIG. 3C depict results from characterization of purified IgG with common light chain (CLC). Purified anti-CD203c IgG-null antibodies with the anti-c-kit MH1 light chain were titrated (in nM) in a direct binding enzyme-linked immunosorbent assay (ELISA) against purified recombinant human (FIG. 3A) and rhesus (FIG. 3B) CD203c proteins. 1.27 was subsequently titrated in a flow-based assay on the basophilic cell line KU812 which expresses CD203c on the cell surface (FIG. 3C).

FIG. 4 depicts the design of the 1.27 heavy chain CDR1 & CDR2 variant optimization library. The heavy chain amino acid sequence for the parental clone 1.27 is shown. CDR1 and CDR2 sequences are depicted in grey. This CDR definition, used throughout the text, is expanded compared to the classical Kabat nomenclature. CDR positions targeted with mutagenesis are highlighted in parentheses. The table illustrates that each targeted position is replaced by any other amino acid (except cysteine) and all possible single and double mutations are sampled.

FIG. 5 depicts the design of the 1.27 heavy chain CDR3 variant optimization library. The heavy chain amino acid sequence for the parental clone 1.27 is shown. The CDR3 sequence is depicted in grey, and CDR3 positions targeted for mutagenesis are highlighted in parentheses. The table illustrates that each targeted position is replaced by any other amino acid (except cysteine) and all possible single and double mutations are sampled.

FIG. 6A-FIG. 6C depict results from characterization of antibody variants from 1.27 optimization libraries. Antibody variants isolated from the 1.27 optimization libraries were compared to the parental 1.27 clone for binding to human (FIG. 6A) and rhesus (FIG. 6B) CD203c by direct binding ELISA, and for binding to KU812 cells by flow-based assay (FIG. 6C).

FIG. 7 depicts results from an assay of binding of CLC bispecifics to KU812 cells. Bispecific molecules composed of anti-c-Kit MH1 and anti-CD203c 1.27 (parental) or anti-CD203c F6 (affinity optimized) were compared to anti-c-Kit IgG, anti-c-Kit one-armed antibody and anti-CD203c IgGs for binding to KU812 cells. The bispecific molecules had similar EC50s to the anti-c-Kit IgG but a much higher Bmax due to co-operative binding of both arms on dual target expressing cells.

FIG. 8 depicts results from an assay of inhibition of c-Kit receptor phosphorylation by F6/MH1 bispecific. The F6/MH1 bispecific molecule demonstrated potent inhibition of c-Kit receptor phosphorylation when compared to the one-armed MH1 c-Kit antibody.

FIG. 9A-FIG. 9B depict results from binding assays with alternative common light chains containing CDRs grafted from MH1 paired with CD203c VH domains. The results demonstrate that not all common light chain/heavy chain pairs retain dual target binding. Alternative common light chains containing CDRs grafted from MH1 paired with CD203c VH domains retain c-Kit binding (FIG. 9A) but lose CD203c (ENPP3) binding (FIG. 9B).

FIG. 10 depicts results from an ELISA comparing binding of the F6/MH1 bispecific molecule to human and cynomolgus CD203c. The results demonstrate that the F6/MH1 bispecific molecule retains equivalent binding to both human and cynomolgus CD203c.

FIG. 11A-FIG. 11D depict results from SPR characterization of affinity optimized CD203c VH binding domains. The parental anti-CD203c binding domain 1.27 (FIG. 11A), and affinity optimized variants F6 (FIG. 11B) and F6.12 (FIG. 11C) were prepared as Fabs and assessed for comparative affinity to human and cyno CD203c using single-cycle kinetics. The results are summarized in the accompanying table (FIG. 11D).

FIG. 12A-FIG. 12B depict results from an analysis of binding of affinity optimized CD203c VH binding domain to KU812 cells. The parental anti-CD203c binding domain 1.27

(FIG. 12A) was produced as an IgG and compared to affinity optimized F6 IgG (FIG. 12B) binding to KU812 cells.

FIG. 13 depicts substitution mutations generated from an analysis of pI engineering of the anti-c-Kit and anti-CD203c VH domains. The table summarizes the top 5 double mutant combinations that were selected for MH1 VH (anti-c-Kit) and F6.12 VH (anti-CD203c) domain engineering to maximize pl differences between the heavy chains.

FIG. 14 depicts preparative CEX (cation exchange) and analytical SEC (size exclusion) chromatograms for MH1_A/F6.12_B bispecific. Preparative CEX was used to assess the efficiency of separation of heterodimer and homodimer species during bispecific purification. The table summarizes the peak composition obtained under these conditions. Samples were also analyzed for monomeric purity by SEC.

FIG. 15 depicts preparative CEX and analytical SEC chromatograms for MH1_C/F6.12_D bispecific. Preparative CEX was used to assess the efficiency of separation of heterodimer and homodimer species during bispecific purification. The table summarizes the peak composition obtained under these conditions. Samples were also analyzed for monomeric purity by SEC.

FIG. 16 depicts results from a comparison of c-Kit target binding pre- and post-pI engineering using SPR. A single-cycle kinetic protocol was used to compare bispecific binding to human and cyno c-Kit before (MH1/F6.12) and after heavy chain pI engineering (MH1_C/F6.12_D).

FIG. 17 depicts results from a comparison of CD203c target binding pre-and post-pI engineering using SPR. A single-cycle kinetic protocol was used to compare bispecific binding to human and cyno CD203c before (MH1/F6.12) and after heavy chain pI engineering (MH1_C/F6.12_D).

FIG. 18 depicts results from a comparison of dual target binding pre- and post-pI engineering using SPR. The table summarizes kinetic data obtained using a single-cycle kinetics protocol to compare human and cyno c-Kit and human and cyno CD203c binding for a number of bispecific constructs before (MH1/F6.12) and after (MH1_A/F6.12_B, MH1_A/F6.12_C, MH1_C/F6.12_B & MH1_C/F6.12_D).

FIG. 19A-FIG. 19C depict results from a comparison of binding to KU812 cells pre- and post-pI engineering using flow cytometry. Binding to dual targets expressed on KU812cells was compared for a number of bispecific constructs before (MH1/F6.12) (FIG. 19A) and after (MH1_C/F6.12_B (FIG. 19B) and MH1_C/F6.12_D) (FIG. 19C) pI engineering.

### DETAILED DESCRIPTION

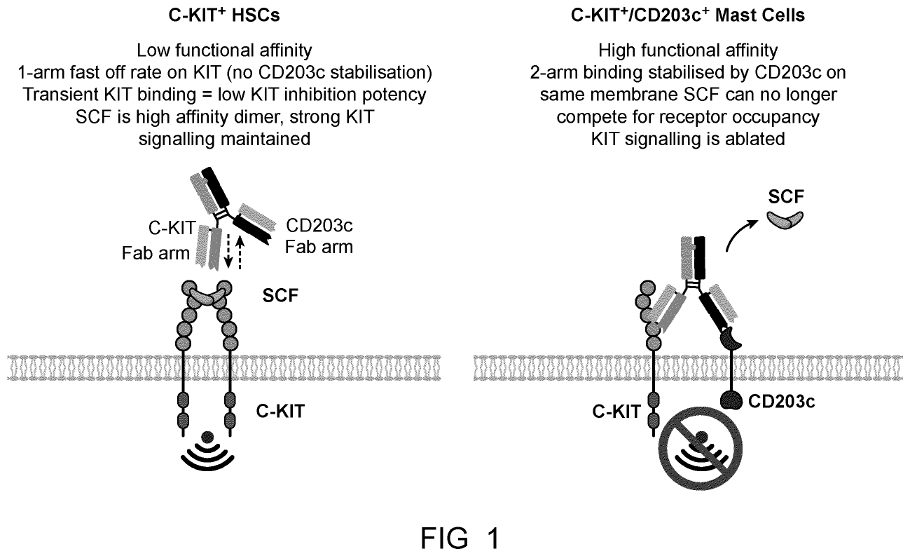

It has been shown that both IgE-dependent and IgE-independent activation of mast cells and basophils leads to a robust upregulation of cell surface CD203c. CD203c is specific to the mast cell/basophil lineage and is not expressed on other hematopoietic stem cell precursors, unlike c-Kit. It has been shown that CD203c differentiates erythroid and granulocyte lineages, therefore, granulocyte targeting via CD203c may avoid myelosuppression. Anti-c-kit/anti-CD203c bispecific antibodies have the potential to maintain potency of mast-cell depletion via c-Kit inhibition, but only on cells that are co-expressing the activated CD203c. This is schematically represented in FIG. 1.

Advances in molecular engineering have enabled the development of bispecific molecules that allows for single agent dosing but is still associated with significant complexity in manufacturing. Issues with correct pairing of both light chains can lead to significant heterogeneity, which requires additional downstream processing. This can be overcome by manufacturing individual antibodies separately with post-production bispecific formation. However, this requires the development of two manufacturing cell lines. The bispecific anti-c-Kit/anti-CD203c proteins described herein use a common light chain format to provide an advantage in streamlined downstream processing.

### Protein Molecules

Provided herein are bispecific proteins that bind c-Kit and CD203c in cis, i.e., wherein the c-Kit and CD203c molecules are on the surface of the same cell.

Provided herein are bispecific proteins comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c.

Provided herein are VL domain sequences that form (1) a binding domain that specifically binds c-Kit when paired with an anti-c-Kit VH domain; and (2) a binding domain that specifically binds CD203c when paired with an anti-CD203c VH domain.

In some embodiments, a protein comprises one or more amino acid sequences provided in Table 1-Table 7.

TABLE 1
Exemplary anti-c-Kit heavy chain binding domain sequences

| | | SEQ |
|---|---|---|
| Name | Sequence | ID NO |
| MH1 VH | QVQLVQSGAEVKKPGASVKVSCKASGYTFTDYYMNWVRQAPGQGLE | 1 |
| domain | WMGRIYPGSGNTYYAQKFQGRVTMTRDTSTSTVYMELSSLRSEDTA | |
| | VYYCARGVYYYDYWGQGTLVTVSS | |
| MH1 | GYTFTDYYMN | 2 |
| HCDR1 | | |
| MH1 | RIYPGSGNTYYA | 3 |
| HCDR2 | | |
| MH1 | ARGVYYYDY | 4 |
| HCDR3 | | |

In the VH domain sequence, the CDR sequences are underlined.

TABLE 2
Exemplary anti-c-Kit/anti-CD203c light chain binding domain sequences

| | | SEQ |
|---|---|---|
| Name | Sequence | ID NO |
| MH1 VL | DIQMTQSPSSLSASVGDRVTITCRASQGIRTNLAWFQQKPGKAPKSLIYA | 5 |
| domain | ASSLQSGVPSRESGSGSGTDFTLTISSLQPEDFATYYCQQYNSYPRTFGG | |
| | GTKVEIK | |
| MH1 | RASQGIRTNLA | 6 |
| LCDR1 | | |
| MH1 | AASSLQSGVPS | 7 |
| LCDR2 | | |
| MH1 | QQYNSYPRT | 8 |
| LCDR3 | | |
| VL1 VL | QSVLTQPPSASGTPGQRATISCRASQGIRTNLAWYQQVPGTAPKLLIYAA | 58 |
| domain | SSLQSGVPSRFSGSKSGTSASLAISGLQSEDEADYYCQQYNSYPRTFGGG | |
| | TKLTVL | |
| VL2 VL | QSVLTQPPSASGTPGQRATISCRASQGIRTNLAWYQQVPGTAPKLLIYSN | 59 |
| domain | NQRPSGVPDRFSGSKSGTSASLAISGLQSEDEADYYCQQYNSYPRTFGGG | |
| | TKLTVL | |
| VL3 VL | EIVLTQSPDFQSVTPKEKVTITCRASQGIRTNLAWYQQKPDQSPKLLIKA | 60 |
| domain | ASSLQSGVPSRFSGSGSGTDFTLTINSLEAEDAATYYCQQYNSYPRTFGQ | |
| | GTKVEIK | |
| VL4 VL | EIVLTQSPDFQSVTPKEKVTITCRASQGIRTNLAWYQQKPDQSPKLLIKY | 61 |
| domain | ASQSFSGVPSRFSGSGSGTDETLTINSLEAEDAATYYCQQYNSYPRTFGQ | |
| | GTKVEIK | |

In the VL domain sequences, the CDR sequences are underlined.

TABLE 3
Exemplary anti-CD203c heavy chain binding domain sequences

| | | SEQ |
|---|---|---|
| Name | Sequence | ID NO |
| 1.27 VH | EVQLVQSGAEVKKPGESLKISCKGSGYRFTSYWIGWVRQMPGKGLEWMGI | 9 |
| domain | IYPGDSDTRYSPSFQGQVTISADKSISTAYLQWSSLKASDTAMYYCARKD | |
| | YYYYAMDVWGQGTTVTVSS | |
| 1.27 | GYRFTSYWIG | 10 |
| HCDR1 | | |
| 1.27 | IIYPGDSDTRYS | 11 |
| HCDR2 | | |
| 1.27 | ARKDYYYYAMDV | 12 |
| HCDR3 | | |
| F6 VH | EVQLVQSGAEVKKPGESLKISCKGSGYRFASNWIGWVRQMPGKGLEWMGE | 13 |
| domain | IYPGDSNTRYSPSFQGQVTISADKSISTAYLQWSSLKASDTAMYYCARKD | |
| | YYYYAMDVWGQGTTVTVSS | |
| F6 | GYRFASNWIG | 14 |
| HCDR1 | | |
| F6 | EIYPGDSNTRYS | 15 |
| HCDR2 | | |
| F6 | ARKDYYYYAMDV | 12 |
| HCDR3 | | |
| C4 VH | EVQLVQSGAEVKKPGESLKISCKGSGYRFGSYWIGWVRQMPGKGLEWMGD | 16 |
| domain | IYPGNSDTRYSPSFQGQVTISADKSISTAYLQWSSLKASDTAMYYCARKD | |
| | YYYYAMDVWGQGTTVTVSS | |
| A5 VH | EVQLVQSGAEVKKPGESLKISCKGSGYRFSSYRIGWVRQMPGKGLEWMGE | 17 |
| domain | IYPGDSKTRYSPSFQGQVTISADKSISTAYLQWSSLKASDTAMYYCARKD | |
| | YYYYAMDVWGQGTTVTVSS | |
| 2 VH | EVQLVQSGAEVKKPGESLKISCKGSGYRFTSYWIGWVRQMPGKGLEWMGI | 18 |
| domain | IYPGDSDTRYSPSFQGQVTISADKSISTAYLQWSSLKASDTAMYYCARHD | |
| | YYYGAMDVWGQGTTVTVSS | |
| 3 VH | EVQLVQSGAEVKKPGESLKISCKGSGYRFTSYWIGWVRQMPGKGLEWMGI | 19 |
| domain | IYPGDSDTRYSPSFQGQVTISADKSISTAYLQWSSLKASDTAMYYCARID | |
| | YYHYAMDVWGQGTTVTVSS | |
| 4 VH | EVQLVQSGAEVKKPGESLKISCKGSGYRFTSYWIGWVRQMPGKGLEWMGI | 20 |
| domain | IYPGDSDTRYSPSFQGQVTISADKSISTAYLQWSSLKASDTAMYYCARQD | |
| | YYYGAMDVWGQGTTVTVSS | |
| 5 VH | EVQLVQSGAEVKKPGESLKISCKGSGYRFTSYWIGWVRQMPGKGLEWMGI | 21 |
| domain | IYPGDSDTRYSPSFQGQVTISADKSISTAYLQWSSLKASDTAMYYCARAD | |
| | EYYYAMDVWGQGTTVTVSS | |
| 6 VH | EVQLVQSGAEVKKPGESLKISCKGSGYRFTSYWIGWVRQMPGKGLEWMGI | 22 |
| domain | IYPGDSDTRYSPSFQGQVTISADKSISTAYLQWSSLKASDTAMYYCARAD | |
| | YLYYAMDVWGQGTTVTVSS | |
| 7 VH | EVQLVQSGAEVKKPGESLKISCKGSGYRFTSYWIGWVRQMPGKGLEWMGI | 23 |
| domain | IYPGDSDTRYSPSFQGQVTISADKSISTAYLQWSSLKASDTAMYYCARND | |
| | YYYGAMDVWGQGTTVTVSS | |
| 8 VH | EVQLVQSGAEVKKPGESLKISCKGSGYRFTSYWIGWVRQMPGKGLEWMGI | 24 |
| domain | IYPGDSDTRYSPSFQGQVTISADKSISTAYLQWSSLKASDTAMYYCARED | |
| | YYYQAMDVWGQGTTVTVSS | |
| 9 VH | EVQLVQSGAEVKKPGESLKISCKGSGYRFTSYWIGWVRQMPGKGLEWMGI | 25 |
| domain | IYPGDSDTRYSPSFQGQVTISADKSISTAYLQWSSLKASDTAMYYCARFD | |
| | IYYYAMDVWGQGTTVTVSS | |
| 10 VH | EVQLVQSGAEVKKPGESLKISCKGSGYRFTSYWIGWVRQMPGKGLEWMGI | 26 |
| domain | IYPGDSDTRYSPSFQGQVTISADKSISTAYLQWSSLKASDTAMYYCARKD | |
| | YYYGAMDVWGQGTTVTVSS | |
| 11 VH | EVQLVQSGAEVKKPGESLKISCKGSGYRFTSYWIGWVRQMPGKGLEWMGI | 27 |
| domain | IYPGDSDTRYSPSFQGQVTISADKSISTAYLQWSSLKASDTAMYYCARKD | |
| | YYYGAFDVWGQGTTVTVSS | |
| 12 VH | EVQLVQSGAEVKKPGESLKISCKGSGYRFTSYWIGWVRQMPGKGLEWMGI | 28 |
| domain | IYPGDSDTRYSPSFQGQVTISADKSISTAYLQWSSLKASDTAMYYCARKD | |
| | YYYGIMDVWGQGTTVTVSS | |
| 13 VH | EVQLVQSGAEVKKPGESLKISCKGSGYRFTSYWIGWVRQMPGKGLEWMGI | 29 |
| domain | IYPGDSDTRYSPSFQGQVTISADKSISTAYLQWSSLKASDTAMYYCARKD | |
| | YYYGSMDVWGQGTTVTVSS | |
| 14 VH | EVQLVQSGAEVKKPGESLKISCKGSGYRFTSYWIGWVRQMPGKGLEWMGI | 30 |
| domain | IYPGDSDTRYSPSFQGQVTISADKSISTAYLQWSSLKASDTAMYYCARKD | |
| | YYYGALDVWGQGTTVTVSS | |
| 15 VH | EVQLVQSGAEVKKPGESLKISCKGSGYRFTSYWIGWVRQMPGKGLEWMGI | 31 |
| domain | IYPGDSDTRYSPSFQGQVTISADKSISTAYLQWSSLKASDTAMYYCARKD | |
| | YYYGTMDVWGQGTTVTVSS | |
| 16 VH | EVQLVQSGAEVKKPGESLKISCKGSGYRFTSYWIGWVRQMPGKGLEWMGI | 32 |
| domain | IYPGDSDTRYSPSFQGQVTISADKSISTAYLQWSSLKASDTAMYYCARKD | |
| | YYEGAMDVWGQGTTVTVSS | |
| 17 VH | EVQLVQSGAEVKKPGESLKISCKGSGYRFTSYWIGWVRQMPGKGLEWMGI | 33 |
| domain | IYPGDSDTRYSPSFQGQVTISADKSISTAYLQWSSLKASDTAMYYCARKD | |
| | YYYNAMPVWGQGTTVTVSS | |
| 18 VH | EVQLVQSGAEVKKPGESLKISCKGSGYRFTSYWIGWVRQMPGKGLEWMGI | 34 |
| domain | IYPGDSDTRYSPSFQGQVTISADKSISTAYLQWSSLKASDTAMYYCARKD | |
| | YYYDAMDYWGQGTTVTVSS | |
| 19 VH | EVQLVQSGAEVKKPGESLKISCKGSGYRFTSYWIGWVRQMPGKGLEWMGI | 35 |
| domain | IYPGDSDTRYSPSFQGQVTISADKSISTAYLQWSSLKASDTAMYYCARKD | |
| | YTYYAMDVWGQGTTVTVSS | |
| 20 VH | EVQLVQSGAEVKKPGESLKISCKGSGYRFTSYWIGWVRQMPGKGLEWMGI | 36 |
| domain | IYPGDSDTRYSPSFQGQVTISADKSISTAYLQWSSLKASDTAMYYCARKD | |
| | YYYSADDVWGQGTTVTVSS | |
| 21 VH | EVQLVQSGAEVKKPGESLKISCKGSGYRFTSYWIGWVRQMPGKGLEWMGI | 37 |
| domain | IYPGDSDTRYSPSFQGQVTISADKSISTAYLQWSSLKASDTAMYYCARKD | |
| | EYYAAMDVWGQGTTVTVSS | |
| 22 VH | EVQLVQSGAEVKKPGESLKISCKGSGYRFTSYWIGWVRQMPGKGLEWMGI | 38 |
| domain | IYPGDSDTRYSPSFQGQVTISADKSISTAYLQWSSLKASDTAMYYCARKD | |
| | EYDYAMDVWGQGTTVTVSS | |
| 23 VH | EVQLVQSGAEVKKPGESLKISCKGSGYRFGSYWIGWVRQMPGKGLEWMGE | 39 |
| domain | IYPGDSKTRYSPSFQGQVTISADKSISTAYLQWSSLKASDTAMYYCARKD | |
| | YYYYAMDVWGQGTTVTVSS | |
| 24 VH | EVQLVQSGAEVKKPGESLKISCKGSGYRFRSYWIGWVRQMPGKGLEWMGI | 40 |
| domain | IYSGDSDTKYSPSFQGQVTISADKSISTAYLQWSSLKASDTAMYYCARKD | |
| | YYYYAMDVWGQGTTVTVSS | |
| 25 VH | EVQLVQSGAEVKKPGESLKISCKGSGYRFASTWIGWVRQMPGKGLEWMGI | 41 |
| domain | IYPGDSQTRYRPSFQGQVTISADKSISTAYLQWSSLKASDTAMYYCARKD | |
| | YYYYAMDVWGQGTTVTVSS | |
| 26 VH | EVQLVQSGAEVKKPGESLKISCKGSGYRFASHWIGWVRQMPGKGLEWMGE | 42 |
| domain | IYPGDSDTRISPSFQGQVTISADKSISTAYLQWSSLKASDTAMYYCARKD | |
| | YYYYAMDVWGQGTTVTVSS | |
| 27 VH | EVQLVQSGAEVKKPGESLKISCKGSGYTFTSYFIGWVRQMPGKGLEWMGI | 43 |
| domain | IYPGDSDSRRSPSFQGQVTISADKSISTAYLQWSSLKASDTAMYYCARKD | |
| | YYYYAMDVWGQGTTVTVSS | |
| 28 VH | EVQLVQSGAEVKKPGESLKISCKGSGVRFTSYDIGWVRQMPGKGLEWMGI | 44 |
| domain | IYTGDSDTRVSPSFQGQVTISADKSISTAYLQWSSLKASDTAMYYCARKD | |
| | YYYYAMDVWGQGTTVTVSS | |
| 29 VH | EVQLVQSGAEVKKPGESLKISCKGSGYRFTSEWIGWVRQMPGKGLEWMGI | 45 |
| domain | IYTGDSDTKYSPSFQGQVTISADKSISTAYLQWSSLKASDTAMYYCARKD | |
| | YYYYAMDVWGQGTTVTVSS | |
| 30 VH | EVQLVQSGAEVKKPGESLKISCKGSGYRFISYWIGWVRQMPGKGLEWMGI | 46 |
| domain | IWPGDSDTRYQPSFQGQVTISADKSISTAYLQWSSLKASDTAMYYCARKD | |
| | YYYYAMDVWGQGTTVTVSS | |
| 31 VH | EVQLVQSGAEVKKPGESLKISCKGSGSRFTSYWIRWVRQMPGKGLEWMGI | 47 |
| domain | IYPGDSDTRFGPSFQGQVTISADKSISTAYLQWSSLKASDTAMYYCARKD | |
| | YYYYAMDVWGQGTTVTVSS | |
| 32 VH | EVQLVQSGAEVKKPGESLKISCKGSGYKFTSDWIGWVRQMPGKGLEWMGI | 48 |
| domain | INPGDSDTRYSPSFQGQVTISADKSISTAYLQWSSLKASDTAMYYCARKD | |
| | YYYYAMDVWGQGTTVTVSS | |
| F6.7 VH | EVQLVQSGAEVKKPGESLKISCKGSGYRFASNWIGWVRQMPGKGLEWMGE | 49 |
| domain | IYPGDSNTRYSPSFQGQVTISADKSISTAYLQWSSLKASDTAMYYCARND | |
| | YYYGAMDVWGQGTTVTVSS | |
| F6.10 | EVQLVQSGAEVKKPGESLKISCKGSGYRFASNWIGWVRQMPGKGLEWMGE | 50 |
| VH | IYPGDSNTRYSPSFQGQVTISADKSISTAYLQWSSLKASDTAMYYCARKD | |
| domain | YYYGAMDVWGQGTTVTVSS | |
| F6.12 | EVQLVQSGAEVKKPGESLKISCKGSGYRFASNWIGWVRQMPGKGLEWMGE | 51 |
| VH | IYPGDSNTRYSPSFQGQVTISADKSISTAYLQWSSLKASDTAMYYCARKD | |
| domain | YYYGIMDVWGQGTTVTVSS | |
| 2.7_1 | EVQLVESGPGLVKPSETLSLTCTVSGGSISSSSYYWGWIRQPPGKGLEWI | 54 |
| VH | GSMYYSGSTYHNPSLKSRVIISVDTSKNQFSLKLSSVTAADTAVYYCARH | |
| domain | YITVAGIFDYWGQGTLVTVSS | |
| 2.7_1.2 | EVQLVESGPGLVKPSETLSLTCTVSGGSISSSSYYWGWIRQPPGKGLEWI | 55 |
| VH | GSMYYSGSTYHNPSLKSRVIISVDTSKNQFSLKLSSVTAADTAVYYCARH | |
| domain | YITVAGIFDYWGQGTLVTVSS | |
| 2.14_1 | QVQLQESGPGLVKPSQTLSLTCTVSGGSISSGGYYWSWIRQHPGKGLEWI | 56 |
| VH | GIIYYSGSTYYNPSLKSRVTISVDTSKNQFSLKLNSVTAADTAVFYCARV | |
| domain | AIVTTIPGGMDVWGQGTTVTVSS | |
| 2.14_1.2 | QVQLQESGPGLVKPSQTLSLTCTVSGGSISSGGYYWSWIRQHPGKGLEWI | 57 |
| VH | GIIYYSGSTYYNPSLKSRVTISVDTSKNQFSLKLNSVTAADTAVFYCARV | |
| domain | AIVTTIPGGMDVWGQGTTVTVSS | |

In the VH domain sequences, the CDR sequences are underlined. Residues in bold font are changed from corresponding residues in the 1.27 sequence.

TABLE 4
Exemplary Fc sequences

| | | SEQ |
|---|---|---|
| Name | Sequence | ID NO |
| Fc knob | ASTKGPSVFPLAPSSKSTSGGTAALGCLVKDYFPEPVTVSWNSGALTSGV | 52 |
| sequence | HTFPAVLQSSGLYSLSSVVTVPSSSLGTQTYICNVNHKPSNTKVDKKVEP | |
| | KSCDKTHTCPPCPAPEAAGAPSVFLFPPKPKDTLMISRTPEVTCVVVDVS | |
| | HEDPEVKFNWYVDGVEVHNAKTKPREEQYNSTYRVVSVLTVLHQDWINGK | |
| | EYKCKVSNKALPAPIEKTISKAKGQPREPQVYTLPPCRDELTKNQVSLWC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| Fc hole | ASTKGPSVFPLAPSSKSTSGGTAALGCLVKDYFPEPVTVSWNSGALTSGV | 53 |
| sequence | HTFPAVLQSSGLYSLSSVVTVPSSSIGTQTYICNVNHKPSNTKVDKKVEP | |
| | KSCDKTHTCPPCPAPEAAGAPSVFLFPPKPKDTLMISRTPEVTCVVVDVS | |
| | HEDPEVKENWYVDGVEVHNAKTKPREEQYNSTYRVVSVLTVLHQDWLNGK | |
| | EYKCKVSNKALPAPIEKTISKAKGQPREPQVCTLPPSRDELTKNQVSLSC | |
| | AVKGFYPSDIAVEWESNGQPENNYKTTPPVLDSDGSFFLVSKLTVDKSRW | |
| | QQGNVFSCSVMHEALHNHYTQKSLSLSPGK | |

The anti-c-Kit/anti-CD203c protein design may be based on sequences derived from IgG1, IgG2, IgG3, IgG4, IgE, IgM, or IgA and may or may not have effector function capacity.

In some embodiments, proteins disclosed herein comprise domains and regions of antibody molecules. The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. IgG, IgA, and IgD constant regions comprise a flexible hinge region between the CH1 domain and the CH2 domain. Each light chain comprises a light chain variable domain (abbreviated herein as VL domain) and a light chain constant region. The light chain constant region comprises one domain, CL. The

VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kabat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins. In some embodiments, a bispecific protein provided herein comprises an Fc region.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

As used herein, the terms “immunological binding” and “immunological binding properties” refer to the non-covalent interactions of the type which occur between an immunoglobulin molecule (e.g., antibody or antigen-binding portion thereof), or a protein comprising an immunoglobulin-derived binding domain(s) and an antigen for which the immunoglobulin or protein is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind PD-L1 or CD3 when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art. One method for determining the K_{d} of an antibody is by using surface plasmon resonance (SPR), typically using a biosensor system such as a Biacore® system.

Functionally, the binding affinity of a protein provided herein may be within the range of 10⁻⁵M to 10⁻¹² M. For example, the binding affinity of a protein provided herein is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10−7 M to 10⁻¹⁰ M, from 10⁻⁸ M to 10⁻¹⁰ M, from 10⁻⁹M to 10⁻¹⁰ M, from 10⁻⁵ M to 10⁻⁹ M, from 10⁻⁶ M to 10⁻⁹M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is a bispecific protein comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c, wherein the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 73; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 10, a HCDR2 comprising the amino acid sequence of SEQ ID NO: 11, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

TABLE 5
Exemplary anti-c-Kit heavy chain binding domain sequences
(MH1 VH domain variants)

| | | SEQ |
|---|---|---|
| Name | Sequence | ID NO |
| MH1_A | EVQLVQSGAEVKKPGASVKVSCKASGYTFTDYYMNWVRQAPGQGLE | 63 |
| | WMGRIYPGSGNTYYAQKFQDRVTMTRDTSTSTVYMELSSLRSEDTA | |
| | VYYCARGVYYYDYWGQGTLVTVSS | |
| MH1_B | QVQLVQSGAEVKEPGASVKVSCKASGYTFTDYYMNWVRQAPGQGLE | 64 |
| | WMGRIYPGSGNTYYAEKFQGRVTMTRDTSTSTVYMELSSLRSEDTA | |
| | VYYCARGVYYYDYWGQGTLVTVSS | |
| MH1_C | QVQLVQSGAEVKEPGASVKVSCKASGYTFTDYYMNWVRQAPGQGLE | 65 |
| | WMGRIYPGSGNTYYAQNFQGRVTMTRDTSTSTVYMELSSLRSEDTA | |
| | VYYCARGVYYYDYWGQGTLVTVSS | |
| MH1_D | QVQLVQSGAEVKEPGASVKVSCKASGYTFTDYYMNWVRQAPGQGLE | 66 |
| | WMGRIYPGSGNTYYAQKFEGRVTMTRDTSTSTVYMELSSLRSEDTA | |
| | VYYCARGVYYYDYWGQGTLVTVSS | |
| MH1_E | QVQLVQSGAEVKEPGASVKVSCKASGYTFTDYYMNWVRQAPGQGLE | 67 |
| | WMGRIYPGSGNTYYAQKFQDRVTMTRDTSTSTVYMELSSLRSEDTA | |
| | VYYCARGVYYYDYWGQGTLVTVSS | |

The CDR sequences are underlined. Residues in bold font are changed from corresponding residues in the MH1 VH domain sequence.

TABLE 6
Exemplary anti-CD203c heavy chain binding domain sequences
(F6.12 VH domain variants)

| | | SEQ |
|---|---|---|
| Name | Sequence | ID NO |
| F6.12_A | QVQLVQSGAEVKKPGGSLKISCKGSGYRFASNWIGWVRQMPGKGLE | 68 |
| | WMGEIYPGDSNTRYSPSFQGQVTISADKSISTAYLQWSSLKASDTA | |
| | MYYCARKDYYYGIMDVWGQGTTVTVSS | |
| F6.12_B | QVQLVQSGAEVKKPGESLKISCKGSGYRFASNWIGWVRQMPGKGLQ | 69 |
| | WMGEIYPGDSNTRYSPSFQGQVTISADKSISTAYLQWSSLKASDTA | |
| | MYYCARKDYYYGIMDVWGQGTTVTVSS | |
| F6.12_C | QVQLVQSGAEVKKPGESLKISCKGSGYRFASNWIGWVRQMPGKGLE | 70 |
| - | WMGEIYPGDSNTRYRPSFQGQVTISADKSISTAYLQWSSLKASDTA | |
| | MYYCARKDYYYGIMDVWGQGTTVTVSS | |
| F6.12_D | QVQLVQSGAEVKKPGESLKISCKGSGYRFASNWIGWVRQMPGKGLE | 71 |
| | WMGEIYPGDSNTRYSPSFQGQVTISADKSISTAYLQWSRLKASDTA | |
| | MYYCARKDYYYGIMDVWGQGTTVTVSS | |
| F6.12_E | QVQLVQSGAEVKKPGESLKISCKGSGYRFASNWIGWVRQMPGKGLE | 72 |
| | WMGEIYPGDSNTRYSPSFQGQVTISADKSISTAYLQWSSLKASDTA | |
| | MYYCARKDYYYGIMDVWGKGTTVTVSS | |

The CDR sequences are underlined. Residues in bold font are changed from corresponding residues in the F6.12 VH domain sequence.

TABLE 7
Sequences of bispecific molecule MH1_C/F6.12_D

| | | SEQ |
|---|---|---|
| Name | Sequence | ID NO |
| MH1_C | QVQLVQSGAEVKEPGASVKVSCKASGYTFTDYYMNWVRQAPGQGLEWM | 65 |
| (anti-c-KIT | GRIYPGSGNTYYAQNFQGRVTMTRDTSTSTVYMELSSLRSEDTAVYYC | |
| VH domain) | ARGVYYYDYWGQGTLVTVSS | |
| anti-c-KIT | GYTFTDYYMN | 2 |
| HCDR1 | | |
| anti-c-KIT | RIYPGSGNTYYA | 3 |
| HCDR2 | | |
| anti-c-KIT | ARGVYYYDY | 4 |
| HCDR3 | | |
| F6.12_D | QVQLVQSGAEVKKPGESLKISCKGSGYRFASNWIGWVRQMPGKGLEWM | 71 |
| (anti-CD203c | GEIYPGDSNTRYSPSFQGQVTISADKSISTAYLQWSRLKASDTAMYYC | |
| VH domain) | ARKDYYYGIMDVWGQGTTVTVSS | |
| anti-CD203c | GYRFASNWIG | 14 |
| HCDR1 | | |
| anti-CD203c | EIYPGDSNTRYS | 15 |
| HCDR2 | | |
| anti-CD203c | ARKDYYYGIMDV | 73 |
| HCDR3 | | |
| MH1 VL | DIQMTQSPSSLSASVGDRVTITCRASQGIRTNLAWFQQKPGKAPKSLI | 5 |
| domain | YAASSLQSGVPSRFSGSGSGTDFTLTISSLQPEDFATYYCQQYNSYPR | |
| | TFGGGTKVEIK | |
| MH1 LCDR1 | RASQGIRTNLA | 6 |
| MH1 LCDR2 | AASSLQSGVPS | 7 |
| MH1 LCDR3 | QQYNSYPRT | 8 |

In the variable domains, the CDR sequences are underlined.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 65; the anti-c-CD203c VH domain comprises the amino acid sequence of SEQ ID NO: 71; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises an amino acid sequence at least about 95%, 96%, 97%, 98%, or 99% identical to the amino acid sequence of SEQ ID NO: 65; the anti-c-CD203c VH domain comprises an amino acid sequence at least about 95%, 96%, 97%, 98%, or 99% identical to the amino acid sequence of SEQ ID NO: 71; and the VL domain comprises an amino acid sequence at least about 95%, 96%, 97%, 98%, or 99% identical to the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 9; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises an amino acid sequence at least about 95%, 96%, 97%, 98%, or 99% identical to the amino acid sequence of SEQ ID NO: 1; the anti-c-CD203c VH domain comprises an amino acid sequence at least about 95%, 96%, 97%, 98%, or 99% identical to the amino acid sequence of SEQ ID NO: 9; and the VL domain comprises an amino acid sequence at least about 95%, 96%, 97%, 98%, or 99% identical to the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 13; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c, wherein the anti-c-Kit VH domain comprises an amino acid sequence at least about 95%, 96%, 97%, 98%, or 99% identical to the amino acid sequence of SEQ ID NO: 1; the anti-c-CD203c VH domain comprises an amino acid sequence at least about 95%, 96%, 97%, 98%, or 99% identical to the amino acid sequence of SEQ ID NO: 13; and the VL domain comprises an amino acid sequence at least about 95%, 96%, 97%, 98%, or 99% identical to the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 51; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c, wherein the anti-c-Kit VH domain comprises an amino acid sequence at least about 95%, 96%, 97%, 98%, or 99% identical to the amino acid sequence of SEQ ID NO: 1; the anti-c-CD203c VH domain comprises an amino acid sequence at least about 95%, 96%, 97%, 98%, or 99% identical to the amino acid sequence of SEQ ID NO: 51; and the VL domain comprises an amino acid sequence at least about 95%, 96%, 97%, 98%, or 99% identical to the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of any one of SEQ ID NOs: 16-51; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable VL domain that is capable of specifically binding c-Kit and CD203c, wherein the anti-c-Kit VH domain comprises the amino acid sequence of any one of SEQ ID NOs: 63-67; the anti-c-CD203c VH domain comprises the amino acid sequence of any one of SEQ ID NOs: 68-72; and the VL domain comprises the amino acid sequence of SEQ ID NO: 5.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 54; and the VL domain comprises the amino acid sequence of SEQ ID NO: 58.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 55; and the VL domain comprises the amino acid sequence of SEQ ID NO: 59.

Provided herein is a bispecific protein comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 56; and the VL domain comprises the amino acid sequence of SEQ ID NO: 60.

Provided herein is a bispecific protein comprising: (a) a first polypeptide chain comprising a first immunoglobulin heavy chain comprising an anti-c-Kit VH domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a VL domain that is capable of specifically binding c-Kit and CD203c, wherein the anti-c-Kit VH domain comprises the amino acid sequence of SEQ ID NO: 1; the anti-c-CD203c VH domain comprises the amino acid sequence of SEQ ID NO: 57; and the VL domain comprises the amino acid sequence of SEQ ID NO: 61.

In some embodiments, an immunoglobulin heavy chain comprises an amino acid sequence provided herein (e.g., in Tables 1, 3 or 5-7), with 1, 2 or 3 conservative amino acid substitutions. In some embodiments, an immunoglobulin light chain comprises an amino acid sequence provided herein (e.g., in Table 2 or Table 7), with 1, 2 or 3 conservative amino acid substitutions. In some embodiments, conservative amino acid substitutions are made only in the FR sequences and not in the CDR sequences.

In some embodiments, a bispecific protein comprises the anti-c-Kit VH domain MH1_C (or the corresponding CDR sequences); the anti-c-CD203c VH domain F6.12_D (or the corresponding CDR sequences); and the VL domain MH1 (or the corresponding CDR sequences).

In some embodiments, a bispecific protein comprises the anti-c-Kit VH domain MH1 (or the corresponding CDR sequences); the anti-c-CD203c VH domain 1.27 (or the corresponding CDR sequences); and the VL domain MH1 (or the corresponding CDR sequences).

In some embodiments, a bispecific protein comprises the anti-c-Kit VH domain MH1 (or the corresponding CDR sequences); the anti-c-CD203c VH domain F6 (or the corresponding CDR sequences); and the VL domain MH1 (or the corresponding CDR sequences).

In some embodiments, a bispecific protein comprises the anti-c-Kit VH domain MH1 (or the corresponding CDR sequences); the anti-c-CD203c VH domain F6.12 (or the corresponding CDR sequences); and the VL domain MH1 (or the corresponding CDR sequences).

In some embodiments, a bispecific protein provided herein comprises an immunoglobulin heavy chain constant region at the C-terminus of an immunoglobulin heavy chain. In some embodiments, the immunoglobulin heavy chain constant region is IgG, IgE, IgM, IgD, IgA or IgY. In some embodiments, the immunoglobulin heavy chain constant region is IgG1, IgG2, IgG3, IgG4, IgA1 or IgA2. In some embodiments, the immunoglobulin heavy chain constant region is IgG1. In some embodiments, the immunoglobulin heavy chain constant region is immunologically inert. In some embodiments, the immunoglobulin heavy chain constant region comprises one or more mutations to reduce or prevent FcγR binding, antibody-dependent cell-mediated cytotoxicity (ADCC) activity, antibody-dependent cellular phagocytosis (ADCP), and/or complement-dependent cytotoxicity (CDC) activity. In some embodiments, the immunoglobulin heavy chain constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kabat. In some embodiments, a position of an amino acid residue in a constant region of an immunoglobulin molecule is numbered according to the EU index as in Kabat (Ward et al., 1995 Therap. Immunol. 2:77-94).

In some embodiments of the proteins provided herein, the first polypeptide chain comprises a first immunoglobulin constant region and the second polypeptide chain comprises a second immunoglobulin constant region, and wherein the first immunoglobulin constant region and the second immunoglobulin constant region comprise knob-in-hole mutations. In some embodiments, the first immunoglobulin constant region comprises a CH3 domain comprising the amino acid substitutions S354C and T366W, and wherein the second immunoglobulin constant region comprises a CH3 domain comprising the amino acid substitutions Y349C, T366S, L368A, and Y407V, wherein numbering is according to the EU index as in Kabat. In some embodiments, the first immunoglobulin constant region comprises a CH3 domain comprising the amino acid substitutions Y349C, T366S, L368A, and Y407V, and wherein the second immunoglobulin constant region comprises a CH3 domain comprising the amino acid substitutions S354C and T366W, wherein numbering is according to the EU index as in Kabat. In some embodiments, (a) the first immunoglobulin constant region comprises the amino acid sequence of SEQ ID NO: 52, and the second immunoglobulin constant region comprises the amino acid sequence of SEQ ID NO: 53; or (b) the first immunoglobulin constant region comprises the amino acid sequence of SEQ ID NO: 53, and the second immunoglobulin constant region comprises the amino acid sequence of SEQ ID NO: 52.

In some embodiments, a protein provided herein may comprise any heterodimerization mutations or heterodimerization technology. In some embodiments, a protein provided herein may not comprise any heterodimerization mutations or heterodimerization technology. In such embodiments, purification techniques may be used to isolate the protein.

In some embodiments, a protein provided herein may comprise an immunoglobulin light chain constant region that is a kappa light chain. In some embodiments a kappa light chain comprises SEQ ID NO: 62.

(SEQ ID NO: 62)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, a protein provided herein may comprise an immunoglobulin light chain constant region that is a lambda light chain.

In some embodiments, a bispecific protein provided herein is a designed ankyrin repeat protein (DARPin). In some embodiments, a bispecific protein provided herein is a tandem VHH. In some embodiments, a bispecific protein provided herein is a tandem immunoglobulin new antigen receptor (IgNAR).

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example, RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

PHARMACEUTICAL COMPOSITIONS

The bispecific proteins provided herein (also referred to herein as “active compounds”) can be incorporated into pharmaceutical compositions suitable for administration. Such compositions typically comprise a protein (or an immunoconjugate comprising said protein), and a pharmaceutically acceptable carrier, diluent or excipient. Such materials should be non-toxic and should not interfere with the efficacy of the protein. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the protein, an increase in its lifespan and/or in its efficacy in the body or an increase in its solubility in solution.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, NJ) or phosphate buffered saline (PBS). In some cases, the composition is sterile and fluid to the extent that easy syringeability exists. In some cases, the composition is stable under the conditions of manufacture and storage and is preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the protein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Proteins

Provided herein is a nucleic acid molecule encoding a bispecific protein disclosed herein. Provided herein is a nucleic acid molecule (e.g., an isolated nucleic acid molecule) encoding the first polypeptide chain, the second polypeptide chain, the third polypeptide chain, and the fourth polypeptide chain of a bispecific protein disclosed herein (or an amino acid sequence of (i) a VH domain, (ii) a VL domain, or (iii) both a VH domain and a VL domain of a protein). In some embodiments, a nucleic acid molecule encoding a VH domain, a VL domain, or a polypeptide chain comprises a signal sequence (or encodes a leader peptide). In some embodiments, a nucleic acid molecule encoding a VH domain, a VL domain, or a polypeptide chain does not comprise a signal sequence (or does not encode a leader peptide).

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding the first polypeptide chain, the second polypeptide chain, the third polypeptide chain, and the fourth polypeptide chain of a bispecific protein disclosed herein. In some embodiments, a recombinant host cell comprises multiple vectors or multiple nucleic acid molecules encoding the first polypeptide chain, the second polypeptide chain, the third polypeptide chain, and the fourth polypeptide chain of a bispecific protein disclosed herein.

Protein molecules of the invention, or portions thereof, can be produced using techniques known in the art, for example, recombinant technologies, phage display technologies, synthetic technologies, computational technologies or combinations of such technologies or other technologies readily known in the art.

Further provided herein is a method for producing a protein disclosed herein, the method comprising: culturing a recombinant host cell comprising an expression vector described herein under conditions whereby the nucleic acid molecule is expressed, thereby producing the protein. The protein may then be isolated from the host cell or culture. Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Proteins disclosed herein can be produced by any of a variety of methods known to those skilled in the art. In certain embodiments, proteins disclosed herein can be produced recombinantly. For example, nucleic acid sequences encoding one or more of the heavy chains or light chains provided herein, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, light chain proteins and heavy chain proteins are produced in a cell with a signal sequence that is removed upon production of a mature protein disclosed herein.

Those skilled in the art will be able to determine whether a protein comprising a given polypeptide sequence binds to c-Kit protein and/or CD203c protein using standard methodologies, for example, Western blots, ELISA, and the like.

### Medical Uses of Bispecific Proteins

Provided herein are methods and uses of bispecific proteins, immunoconjugates, and pharmaceutical compositions disclosed herein for providing a therapeutic benefit to a subject with an inflammatory disease. Provided herein are methods and uses of bispecific proteins, immunoconjugates, and pharmaceutical compositions disclosed herein for providing a therapeutic benefit to a subject with a neoplasm.

An activatable protein, immunoconjugate, or pharmaceutical composition disclosed herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the protein, immunoconjugate, or pharmaceutical composition to a subject in need thereof.

Provided herein is a method for treating an inflammatory disease or a neoplasm in a subject, the method comprising administering to the subject a therapeutically effective amount of a protein, an immunoconjugate, or a pharmaceutical composition disclosed herein. Provided herein is a bispecific protein, an immunoconjugate, or a pharmaceutical composition disclosed herein for use in the treatment of an inflammatory disease or a neoplasm.

Provided herein is a method for ameliorating a symptom of an inflammatory disease or a neoplasm in a subject, the method comprising administering to the subject a therapeutically effective amount of a protein, immunoconjugate, or a pharmaceutical composition disclosed herein.

Provided herein is a bispecific protein, an immunoconjugate, or a pharmaceutical composition disclosed herein for use as a medicament.

In some embodiments of the methods and uses disclosed herein, the inflammatory disease is a chronic inflammatory disease. In some embodiments of the methods and uses disclosed herein, the inflammatory disease is a mast cell-driven disease. In some embodiments of the methods and uses disclosed herein, the inflammatory disease is chronic urticaria, chronic pruritus, atopic dermatitis, allergic asthma, prurigo nodularis, eosinophilic gastritis, eosinophilic duodenitis, or eosinophilic esophagitis.

In some embodiments of the methods and uses disclosed herein, the neoplasm is a mast cell-driven neoplasm. In some embodiments of the methods and uses disclosed herein, the neoplasm is systemic mastocytosis or mast cell leukemia.

As used herein, the term “effective amount” or “therapeutically effective amount” refers to the amount of a pharmaceutical agent, e.g., a protein, an immunoconjugate, or a pharmaceutical composition disclosed herein, which is sufficient to reduce or ameliorate the severity and/or duration of an inflammatory disease or a neoplasm, or one or more symptoms thereof, prevent the advancement of a disease, cause regression of a disease, prevent the recurrence, development, onset or progression of one or more symptoms associated with a disease, or enhance or improve the prophylactic or therapeutic effect(s) of another related therapy (e.g., prophylactic or therapeutic agent) for an inflammatory disease or a neoplasm.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g., decisions on dosage etc., is within the responsibility of general practitioners and other medical doctors and may depend on the severity of the symptoms and/or progression of a disease being treated. Appropriate doses of antibody-based protein molecules are well known in the art (Ledermann J.A. et al., 1991, Int. J. Cancer 47:659-664; Bagshawe K.D. et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody-based protein molecule may be determined by comparing its in vitro activity and in vivo activity in an animal model. Methods for extrapolation of effective dosages in mice and other test animals to humans are known. The precise dose will depend upon a number of factors, including whether the antibody-based protein is for prevention or for treatment, the size and location of the area to be treated, the precise nature of the antibody-based protein, and the nature of any detectable label or other molecule attached to the antibody-based protein.

A typical protein dose will be in the range 100 μg to 1 g for systemic applications, and 1 μg to 1 mg for intradermal injection. An initial higher loading dose, followed by one or more lower doses, may be administered. In some embodiments, the protein is an IgG1 or IgG4 isotype. A dose for a single treatment of an adult subject may be proportionally adjusted for children and infants. Treatments may be repeated at daily, twice-weekly, weekly or monthly intervals, at the discretion of the physician. The treatment schedule for a subject may be dependent on the pharmacokinetic and pharmacodynamic properties of the protein composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, a protein, an immunoconjugate, or a pharmaceutical composition disclosed herein may be administered as a sub-cutaneous injection. Sub-cutaneous injections may be administered using an auto-injector, for example for long term prophylaxis/treatment.

In some embodiments, the therapeutic effect of a protein, an immunoconjugate, or a pharmaceutical composition disclosed herein may persist for several half-lives, depending on the dose. For example, the therapeutic effect of a single dose of a protein, an immunoconjugate, or a pharmaceutical composition disclosed herein may persist in a subject for 1 month or more, 2 months or more, 3 months or more, 4 months or more, 5 months or more, or 6 months or more.

In some embodiments, a subject may be treated with a protein, an immunoconjugate, or a pharmaceutical composition disclosed herein and an additional therapeutic agent or therapy that is used to treat a cancer or a symptom or complication of an inflammatory disease or a neoplasm. The protein, immunoconjugate, or pharmaceutical composition disclosed herein and the additional therapeutic agent or therapy may be administered simultaneously or sequentially.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

Further provided herein is a protein, an immunoconjugate, or a pharmaceutical composition disclosed herein, for use in the treatment of a disease or a disorder.

Provided herein is a protein, an immunoconjugate, or a pharmaceutical composition disclosed herein, for use as a medicament.

### DEFINITIONS

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

The terms “amino-terminal”, “N-terminus”, “carboxyl-terminal”, and “C-terminus” are used herein to denote positions within polypeptide chains. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence but is not necessarily at the carboxyl-terminus of the complete polypeptide.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The terms “antibody-drug conjugate” and “immunoconjugate” refer to a protein of the disclosure that is conjugated to a cytotoxic, a cytostatic and/or a therapeutic agent.

The term “isolated molecule” (where the molecule is, for example, a protein, a polypeptide, a polynucleotide, an antibody or an antigen-binding molecule) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of methods known in the art. For example, the purity of a polypeptide sample may be assayed using polyacrylamide gel electrophoresis and staining of the gel to visualize the polypeptide. For certain purposes, higher resolution may be provided by using high-performance liquid chromatography (HPLC) or other means known in the art for purification.

c-Kit (also known as KIT, Cluster of Differentiation 117 (CD117), PBT, SCFR, KIT proto-oncogene receptor tyrosine kinase) is a transmembrane protein that belongs to the immunoglobulin superfamily and binds to the soluble factor SCF (stem cell factor). c-Kit is a receptor tyrosine kinase type III that is highly expressed by hematopoietic stem cells as well as multiple other cell types, such as mature mast cells.

CD203c (also known as ENPP3, B10, NPP3, PDNP3, and PD-IBETA) is a type II transmembrane n that belongs to the ectonucleotide pyrophosphatase/phosphosdiesterase 3 (E-NPP3) family of enzymes involved in hydrolysis of oligonucleotides and nucleoside phosphates. CD203c is specific to the mast cell/basophil lineage of hematopoietic effector cells.

The terms “inhibit”, “block”, or “neutralize”, as used herein with respect to bioactivity of a protein disclosed herein means the ability of the protein to substantially antagonize, prohibit, prevent, restrain, slow, disrupt, eliminate, stop, reduce or reverse for example progression, strength, or severity of that which is being inhibited including, but not limited to, the binding of c-Kit to SCF, or the binding of CD203c to a binding partner, including, but not limited to an enzymatic inhibitor.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a protein or a pharmaceutical composition provided herein that is effective to treat a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

In the present description, any concentration range, percentage range, ratio range, or integer range is to be understood to include the value of any integer within the recited range and, when appropriate, fractions thereof (such as one tenth and one hundredth of an integer), unless otherwise indicated.

The use of the alternative (e.g., “or”) should be understood to mean either one, both, or any combination thereof of the alternatives.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

### NUMBERED EMBODIMENTS

Notwithstanding the appended claims, the disclosure sets forth the following numbered embodiments:

Embodiment 1. A bispecific protein comprising: (a) a first polypeptide chain comprising a first immunoglobulin heavy chain comprising an anti-c-Kit heavy chain variable (VH) domain; (b) a second polypeptide chain comprising a second immunoglobulin heavy chain comprising an anti-CD203c VH domain; and (c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c.

Embodiment 2. The bispecific protein of embodiment 1, wherein the anti-c-Kit VH domain comprises a heavy chain complementarity determining region 1 (HCDR1) comprising the amino acid sequence of SEQ ID NO: 2, a heavy chain complementarity determining region 2 (HCDR2) comprising the amino acid sequence of SEQ ID NO: 3, and a heavy chain complementarity determining region 3 (HCDR3) comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 73; and the VL domain comprises a light chain complementarity determining region 1 (LCDR1) comprising the amino acid sequence of SEQ ID NO: 6, a light chain complementarity determining region 2 (LCDR2) comprising the amino acid sequence of SEQ ID NO: 7, and a light chain complementarity determining region 3 (LCDR3) comprising the amino acid sequence of SEQ ID NO: 8.

Embodiment 3. The bispecific protein of embodiment 1, wherein the anti-c-Kit VH domain comprises a HCDR 1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 10, a HCDR2 comprising the amino acid sequence of SEQ ID NO: 11, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8

Embodiment 4. The bispecific protein of embodiment 1, wherein the anti-c-Kit VH domain comprises a HCDR 1 comprising the amino acid sequence of SEQ ID NO: 2, a HCDR2 comprising the amino acid sequence of SEQ ID NO: 3, and a HCDR3 comprising the amino acid sequence of SEQ ID NO: 4; the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 12; and the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

Embodiment 5. The bispecific protein of embodiment 2, wherein the anti-c-Kit VH domain comprises the amino acid sequence of SEQ ID NO: 65; the anti-c-CD203c VH domain comprises the amino acid sequence of SEQ ID NO: 71; and the VL domain comprises the amino acid sequence of SEQ ID NO: 5.

Embodiment 6. The bispecific protein of embodiment 3, wherein the anti-c-Kit VH domain comprises the amino acid sequence of SEQ ID NO: 1; the anti-c-CD203c VH domain comprises the amino acid sequence of SEQ ID NO: 9; and the VL domain comprises the amino acid sequence of SEQ ID NO: 5.

Embodiment 7. The bispecific protein of embodiment 4, wherein the anti-c-Kit VH domain comprises the amino acid sequence of SEQ ID NO: 1; the anti-c-CD203c VH domain comprises the amino acid sequence of SEQ ID NO: 13; and the VL domain comprises the amino acid sequence of SEQ ID NO: 5.

Embodiment 8. The bispecific protein of embodiment 1, wherein the anti-c-Kit VH domain comprises the amino acid sequence of SEQ ID NO: 1; the anti-c-CD203c VH domain comprises the amino acid sequence of any one of SEQ ID NOs: 16-51; and the VL domain comprises the amino acid sequence of SEQ ID NO: 5.

Embodiment 9. The bispecific protein of embodiment 1, wherein the anti-c-Kit VH domain comprises the amino acid sequence of any one of SEQ ID NOs: 63-67; the anti-c-CD203c VH domain comprises the amino acid sequence of any one of SEQ ID NOs: 68-72; and the VL domain comprises the amino acid sequence of SEQ ID NO: 5.

Embodiment 10. The bispecific protein of any one of embodiments 1-9, wherein the bispecific protein comprises an immunoglobulin constant region.

Embodiment 11. The bispecific protein of embodiment 10, wherein the immunoglobulin constant region is an IgG, IgE, IgM, IgD, IgA or IgY immunoglobulin constant region.

Embodiment 12. The bispecific protein of embodiment 10, wherein the immunoglobulin constant region is an IgG1, IgG2, IgG3, IgG4, IgA1 or IgA2 immunoglobulin constant region.

Embodiment 13. The bispecific protein of embodiment 10, wherein the immunoglobulin constant region is an immunologically inert constant region.

Embodiment 14. The bispecific protein of any one of embodiments 1-9, wherein the first polypeptide chain comprises a first immunoglobulin constant region and the second polypeptide chain comprises a second immunoglobulin constant region, and wherein the first immunoglobulin constant region and the second immunoglobulin constant region comprise knob-in-hole mutations.

Embodiment 15. The bispecific protein of embodiment 14, wherein the first immunoglobulin constant region comprises a CH3 domain comprising the amino acid substitutions S354C and T366W, and wherein the second immunoglobulin constant region comprises a CH3 domain comprising the amino acid substitutions Y349C, T366S, L368A, and Y407V, wherein numbering is according to the EU index as in Kabat.

Embodiment 16. The bispecific protein of embodiment 14, wherein the first immunoglobulin constant region comprises a CH3 domain comprising the amino acid substitutions Y349C, T366S, L368A, and Y407V, and wherein the second immunoglobulin constant region comprises a CH3 domain comprising the amino acid substitutions S354C and T366W, wherein numbering is according to the EU index as in Kabat.

Embodiment 17. The bispecific protein of embodiment 14, wherein (a) the first immunoglobulin constant region comprises the amino acid sequence of SEQ ID NO: 52, and the second immunoglobulin constant region comprises the amino acid sequence of SEQ ID NO: 53; or (b) the first immunoglobulin constant region comprises the amino acid sequence of SEQ ID NO: 53, and the second immunoglobulin constant region comprises the amino acid sequence of SEQ ID NO: 52.

Embodiment 18. A bispecific protein that binds c-Kit and CD203c, wherein the c-Kit and CD203c are on the surface of the same cell.

Embodiment 19. The bispecific protein of embodiment 18, wherein the bispecific protein is a designed ankyrin repeat protein (DARPin), a tandem VHH, or a tandem immunoglobulin new antigen receptor (IgNAR).

Embodiment 20. An immunoconjugate comprising the bispecific protein of any one of embodiments 1-19, linked to a therapeutic agent.

Embodiment 21. The immunoconjugate of embodiment 20, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Embodiment 22. A pharmaceutical composition comprising the bispecific protein of any one of embodiments 1-19 or the immunoconjugate of embodiment 20 or 21, and a pharmaceutically acceptable carrier, diluent or excipient.

Embodiment 23. A nucleic acid molecule encoding the first polypeptide chain, the second polypeptide chain, the third polypeptide chain, and the fourth polypeptide chain of the bispecific protein of any one of embodiments 1-17.

Embodiment 24. A nucleic acid molecule encoding the bispecific protein of embodiment 18 or 19.

Embodiment 25. An expression vector comprising the nucleic acid molecule of embodiment 22 or 23.

Embodiment 26. A recombinant host cell comprising the nucleic acid molecule of embodiment 23 or 24 or the expression vector of embodiment 25.

Embodiment 27. A method of producing a bispecific protein, the method comprising: culturing the recombinant host cell of embodiment 26 under conditions whereby the nucleic acid molecule is expressed, thereby producing the protein; and isolating the protein from the host cell or culture.

Embodiment 28. A method for treating an inflammatory disease or a neoplasm in a subject in need thereof, the method comprising administering to the subject a therapeutically effective amount of the bispecific protein of any one of embodiments 1-19, the immunoconjugate of embodiment 20 or 21, or the pharmaceutical composition of embodiment 22.

Embodiment 29. A method for ameliorating a symptom of an inflammatory disease or a neoplasm in a subject in need thereof, the method comprising administering to the subject a therapeutically effective amount of the bispecific protein of any one of embodiments 1-19, the immunoconjugate of embodiment 20 or 21, or the pharmaceutical composition of embodiment 22.

Embodiment 30. The method of embodiment 28 or 29, wherein the inflammatory disease is a chronic inflammatory disease.

Embodiment 31. The method of embodiment 28 or 29, wherein the inflammatory disease is a mast cell-driven disease.

Embodiment 32. The method of embodiment 28 or 29, wherein the inflammatory disease is chronic urticaria, chronic pruritus, atopic dermatitis, allergic asthma, prurigo nodularis, eosinophilic gastritis, eosinophilic duodenitis, or eosinophilic esophagitis.

Embodiment 33. The method of embodiment 28 or 29, wherein the neoplasm is a mast cell-driven neoplasm.

Embodiment 34. The method of embodiment 28 or 29, wherein the neoplasm is systemic mastocytosis or mast cell leukemia.

Embodiment 35. The bispecific protein of any one of embodiments 1-19, the immunoconjugate of embodiment 20 or 21, or the pharmaceutical composition of embodiment 22 for use as a medicament.

Embodiment 36. The bispecific protein of any one of embodiments 1-19, the immunoconjugate of embodiment 20 or 21, or the pharmaceutical composition of embodiment 22 for use in the treatment of an inflammatory disease or a neoplasm.

Embodiment 37. The bispecific protein, the immunoconjugate, or the pharmaceutical composition for use of embodiment 36, wherein the inflammatory disease is a chronic inflammatory disease.

Embodiment 38. The bispecific protein, the immunoconjugate, or the pharmaceutical composition for use of embodiment 36, wherein the inflammatory disease is a mast cell-driven disease.

Embodiment 39. The bispecific protein, the immunoconjugate, or the pharmaceutical composition for use of embodiment 36, wherein the inflammatory disease is chronic urticaria, chronic pruritus, atopic dermatitis, allergic asthma, prurigo nodularis, eosinophilic gastritis, eosinophilic duodenitis, or eosinophilic esophagitis.

Embodiment 40. The bispecific protein, the immunoconjugate, or the pharmaceutical composition for use of embodiment 36, wherein the neoplasm is a mast cell-driven neoplasm.

Embodiment 41. The bispecific protein, the immunoconjugate, or the pharmaceutical composition for use of embodiment 36, wherein the neoplasm is systemic mastocytosis or mast cell leukemia.

The disclosure will be further clarified by the following example, which is intended to be purely exemplary of the disclosure and in no way limiting.

### EXAMPLE

### Generation and Analysis of Anti-c-Kit and Anti-CD203c Bispecific Proteins

### Materials and Methods

### IgG/Bispecific Expression and Purification

Mammalian codon-optimized synthetic genes encoding the heavy and light chain variable domains of the anti-CD203c antibodies were cloned into mammalian expression vectors comprising effector function null human IgG1 (“IgG1null”; human IgG1 containing L234A, L235A, G237A mutations in the lower hinge that abrogate normal immunoglobulin ADCC, ADCP and CDC functions) and human Cκ domains, respectively. Fc sequences are provided in Table 4. Co-transfection of heavy and light chain containing vectors in a CHO mammalian expression system was performed, followed by protein A-based purification of the IgG, quantification and quality control on denaturing and non-denaturing SDS-PAGE and analytical SEC.

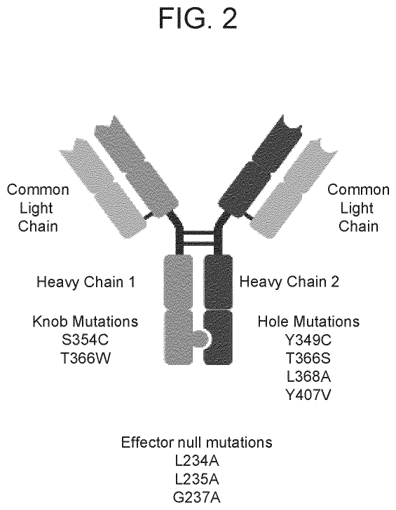

Expression and purification of common light chain bispecific antibodies was carried out in a similar fashion, with heavy chain constructs carrying “knob” or “hole” mutations described in FIG. 2 in addition to effector function null human IgG1 mutations described above. Post protein-A purification, bispecific molecules were subjected to a second step of preparative cation-exchange chromatography to achieve a single POI>95%.

### Direct Binding ELISA for IgG/Bispecifics

Binding and cross-reactivity of the molecules to the human, rhesus, and cynomolgus CD203c was initially assessed by binding ELISA. The human CD203c His-tagged recombinant protein and the rhesus monkey CD203c His-tagged recombinant protein were coated to the surface of MaxiSorp™ flat-bottom 96 well plates at a concentration of 1 μg/ml. The purified samples were titrated in 5-fold serial dilutions starting from 100 nM and allowed to bind to the coated antigens. The IgGs were detected using mouse anti-human IgG conjugated to horseradish peroxidase. Binding signals were visualized with 3,3′,5,5′-Tetramethylbenzidine Substrate Solution (TMB) and the absorbance measured at 450 nm.

### Binding to Cell-Surface CD203c on KU812 Cells

All treatments were performed on cells seeded in T75 flasks at a seeding density of 0.3e6/ml. Cells were cultured in the presence of 80 ng/ml of SCF and treated with 20 ng/ml of IL3 24 hours prior to antibody staining. Following SCF/IL3 treatment, cells were harvested and stained with live dead stain (Zombie UV Biolegend 423108). Cells were then stained with a titration of anti-CD203c antibodies/bispecifics or respective isotype controls in a 5-fold dilution series starting at 50 μg/mL and detected with Anti human-Fc AF647 antibody (JIR 109-605-098). Samples were subsequently run on a Yeti analyzer, gating on 10K live cells. Subsequent analysis was performed using FlowJo software and data represented as MFI (median fluorescent intensity).

### 1.27 Heavy Chain Optimization Library Design, Cloning and Selection

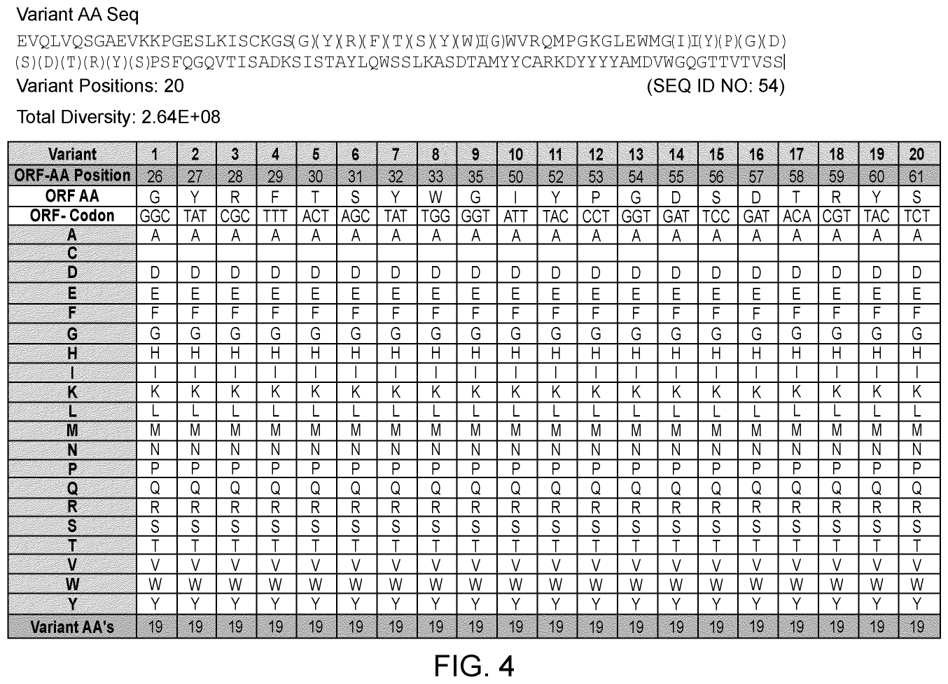

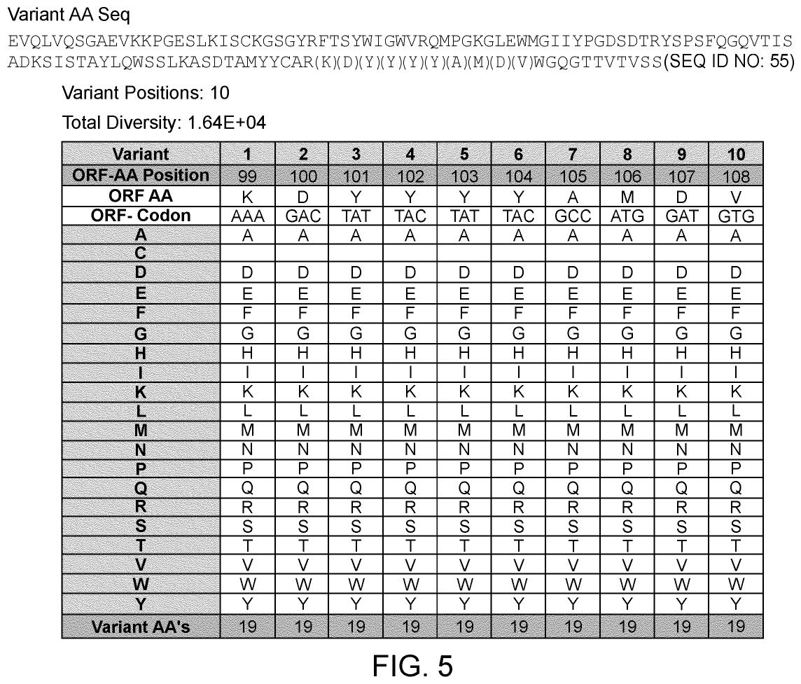

One 2-domain (HCDR1 & HCDR2) precision defined complexity variant library with single and double mutations and one single-domain (HCDR3) precision defined complexity variant library with single and double mutations were synthesized using mass oligo assembly and high-fidelity oligo pool splicing. All amino acids were represented at all positions except cysteine, with insertion of NG and DG developability risk motifs being avoided. These designs are summarized in FIG. 4 and FIG. 5. These libraries were cloned into a custom pCAT-Fab-MH1 phagemid vector containing the anti-c-Kit-MH1 VL, transformed into E. coli TG-1 cells and rescued essentially as previously described in detail (Finlay et al., 2011, Methods Mol Biol 681:383-401).

Phage selections were performed by coating streptavidin magnetic microbeads with biotinylated CD203c protein (either human or rhesus), washing the beads thrice with PBS and resuspending in PBS pH7.4 containing 5% skim milk protein. These beads were coated at 100 nM target protein in round 1 of selection, followed by 4-5-fold reduction in antigen concentrations in three successive rounds. In each round, phage were eluted using trypsin before re-infection into TG1 cells.

### Periplasmic Extracts Production (Small-Scale)

Production of soluble Fabs for individual E. coli clones was performed. E. coli TG1 cells in logarithmic growth phase were induced with isopropyl 1-thio-β-D-galactopyranoside. Periplasmic extracts containing soluble Fab were generated by a freeze/thaw cycle: Bacterial cell pellets were frozen at −20° C. for overnight and then thawed at room temperature and resuspended in PBS pH 7.4. The supernatants containing the soluble Fab were collected after shaking at room temperature and centrifugation. These were tested for binding to 1 μg/mL coated human/rhesus CD203c in direct binding ELISA starting at 85% periprep with a 1.5-fold dilution over an 8-point titration. Anti-HA-HRP was used to detect periprep binding. C-kit receptor phosphorylation assay

KU812 cells were cultured in SCF-free RPMI +10% FCS for 48 h. Cells were seeded in 24-well plates at 1×10⁶/mL in a total volume of 1.5 mL for 1 h, followed by treatment with isotype control or test agents for 2 h. Cells were stimulated with SCF at 40 ng/ml for 5 min, washed in PBS and cell lysates were prepared using lysis buffer according to manufacturer's instructions (Biotechne). Total and phospho-c-kit levels were detected using 6.25 μg and 50 μg total protein, respectively, using ELISA kits from Biotechne.

### Affinity Determination for Optimized CD203c Binding Domains Using SPR

Post-affinity optimization, the CD203c VH binding domains F6 & F6.12 were produced as Fab and compared to parental 1.27 Fab binding to human and cynomolgus monkey CD203c proteins. Biotinylated versions of the CD203c proteins were captured on a BiotinCap chip, and the antibodies injected in-solution in 2-fold serial dilutions. An SPR affinity determination assay was carried out on a BIAcore T20 using the conditions described below:
Biacore Chip: CAP chip (Biotin CAPture kit; Cytiva; Cat. nr. 28920234)
Surface Preparation: Biotin CAPture reagent (Biotin CAPture kit; Cytiva; Cat. nr. 28920234) injected for 5 min at 2 μl/min
Ligand Capture—biotinylated antigen: bio-huCD203c (Acro, Cat. nr. H52H4) and bio-cyCD203c (Icosagen) at 20 nM and 10 nM, respectively, in 1×HBS-EP+pH 7.4, on flow cell 2 over 1 min at 10 μl/min
Analyte Binding: Three test antibodies in eight step two-fold dilution series (1000 nM-7.81 nM for 1.27-Fab, F6 Fab and F6.12 Fab), were injected in flow-cells 1 and 2 for 1 min at 30 μl/min. Complete blank assay runs without analyte antibody were included as blank reference.
Off-rate measurement: 600 seconds
Regeneration: Baseline levels were restored using the regeneration solutions provided in the Biotin CAPture kit
SPR running buffer: 1×HBS-EP+pH 7.4 (Cytiva; cat. nr. BR100669)
Analysis: Data was analyzed using the Biacore Insight Evaluation Software by the steady state affinity model or the 1:1 binding model. Sensorgrams were referenced to flow cell 1 and the 0 nM concentration cycle.

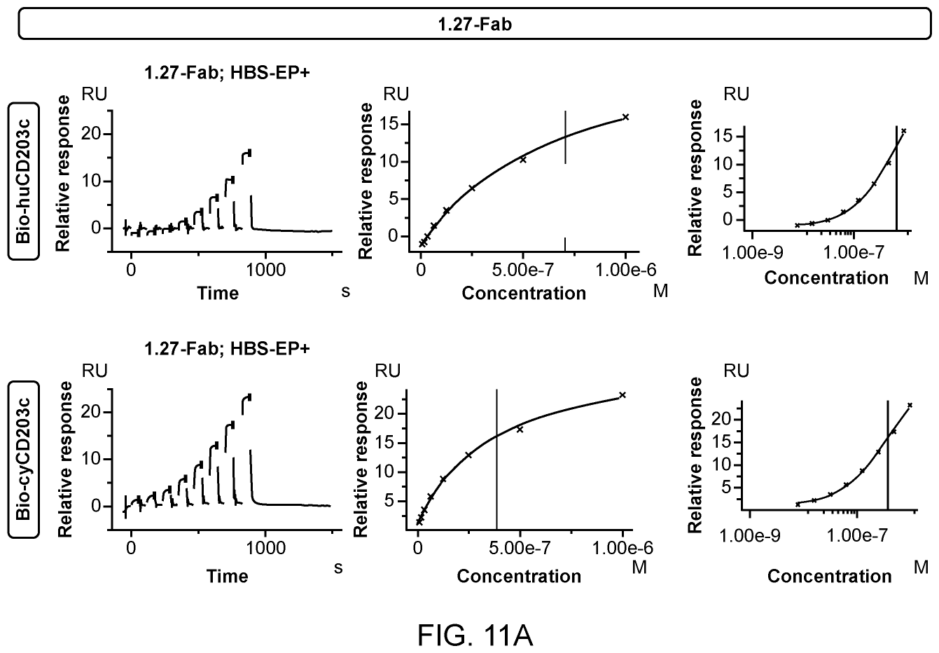

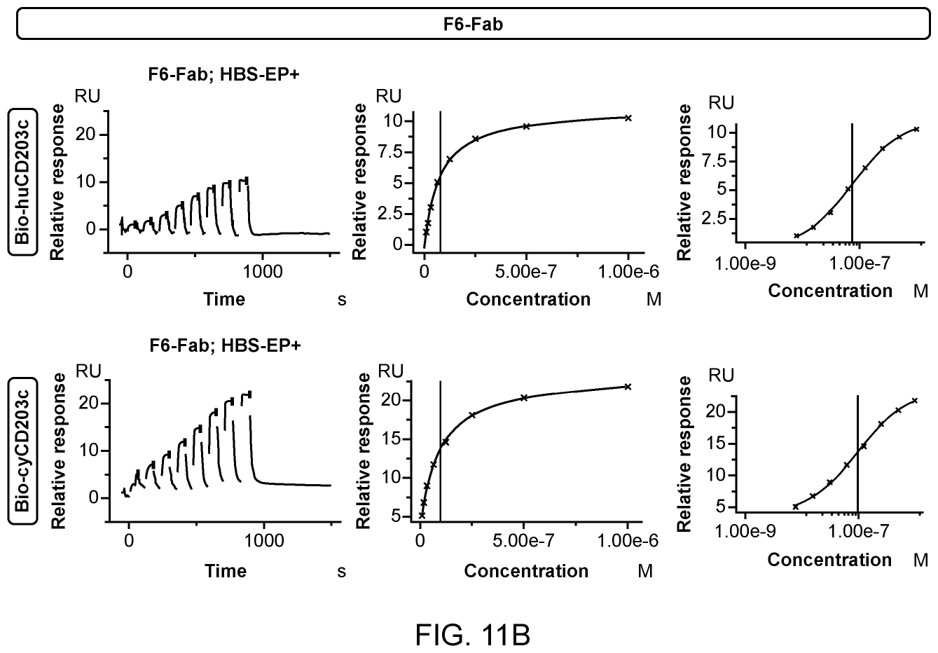

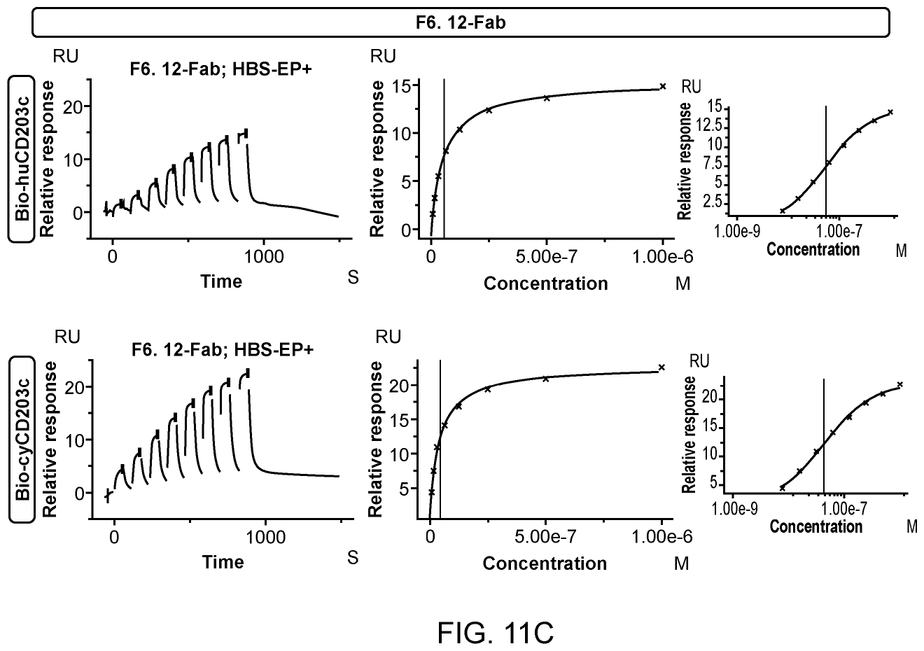

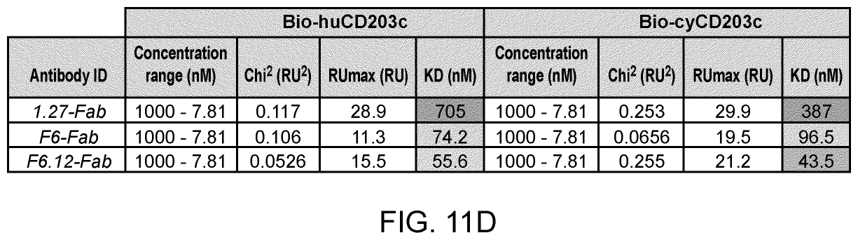

The data for the three Fabs are summarized in FIG. 11A-FIG. 11D.

### pI Engineering of Bispecific Heavy Chains to Enable Facile Purification

The MH1 anti-c-Kit heavy chain & F6.12 anti-CD203c heavy chain were selected for pI engineering to enable facile downstream purification post-ProteinA chromatography using cation-exchange. A number of criteria were used to select mutations driving towards a more negative pI for the anti-c-Kit heavy chain, and a more positive pI for the anti-CD203c heavy chain. These included:
Distal to antigen-interacting CDRs
Analysis of available NGS antibody repertoires in the published literature
Alignment to germline homologs (VH1-46 for anti-c-Kit/VH5-51) for anti-CD203c
Comparison to known and approved clinical stage antibody therapeutics
Comparison against the deposited structures for the 2 germlines in the PDB to give structural context to the selected mutations
All mutant candidates were modelled for predicted impact on stability, dipole moment, developability and immunogenicity

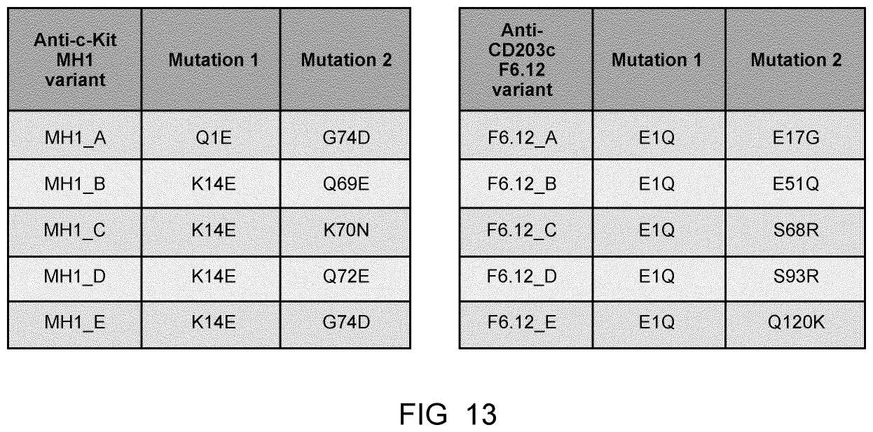

A series of 5 double mutants were prioritized for each heavy chain which had improved, or neutral stability change while changing pI. These double mutants were not present within the same 12-mer sequence stretch to reduce immunogenicity risk and did not create adverse charge/hydrophobicity patches or post-translational modification sites. They maintained developability parameters within, or close to, those described for clinical stage therapeutic antibodies. These double mutation combinations are summarized in FIG. 13.

Production, Purification and Characterization of Lead pI-Engineered Bispecific Antibodies

The 5 double mutants of the MH1 anti-c-Kit VH domain were combined with each of the 5 double mutants of the F6.12 CD203c VH domain to produce 25 bispecifics which were each produced in 50 mL of ExpiCHO cells. ExpiCHO cells were cultured for 9 days, depending on cell viability, after which they were centrifuged to clarify the supernatants and processed immediately. Produced antibodies were captured from clarified supernatants using a Hi Trap MabSelect Sure Protein A 5mL column (GE Healthcare, Cat. nr. 11-0034-95) on an ÄKTA Pure 25 FPLC system. Acidic fractions corresponding to affinity purified antibodies were immediately neutralized with 30% of 1 M Tris pH 8.0. 2.5 μL of each sample was analyzed using a Protein clear HT chip (Cat. nr. CLS1486695) on LabChip under non-reducing and reducing conditions, following the manufacturer's instructions. Approximately 10 μg of each sample was analyzed by SEC-HPLC on a Thermo Vanquish Flex UHPLC system (Thermo Fisher). The sample was injected onto a Superdex 200 Increase 5/150 GL (Cat. nr. 28-9909-45) column and % monomeric peak was quantified. In order to carry out an initial assessment of peak separation via cation-exchange, analytical CEX was carried out for each sample. Approximately 10 μg of each sample was injected onto a TSKgel SP-STAT (7 μm, 4.6 mm ID ×10 cm L) column previously equilibrated in CEX Buffer A (25 mM Sodium Phosphate (pH 6). An increasing salt gradient was applied using CEX Buffer B (25 mM Sodium Phosphate, 1M sodium chloride (pH 6).

For further characterization of prioritized bispecifics, the same process was repeated using a scaled-up volume of 200 mL of ExpiCHO cells. Post Pro-A purification, a number of different preparative cation-exchange methods were compared to assess optimal resolution for the main heterodimeric bispecific peak. Initially, desalted sample was injected into a HiTrap SP HP 5 mL CEX column (Cytiva, cat. nr. 17-1151-02). A total of 2 runs were performed, in order to not compromise resolution of the separation. A salt gradient from 1-20% buffer B (25 mM NaH₂PO₄/Na₂HPO₄ 1 M NaCl pH 6.3) in 40CV, followed by 20-100% of buffer B in 10CV was tested. However, the resolution of the column was not optimal and the RESOURCE S 6 mL CEX column (Cytiva, cat. nr. 17118001) was selected as an alternative using 5-12% buffer B in 40CV, followed by 12-100% of buffer B in 10CV. Several column fractions were taken for analytical SDS-PAGE characterization by LabChip, as described above, and LC-MS analysis. Each sample was deglycosylated using Rapid PNGase F (non-reducing format) (New England Biolabs, Cat. nr. PO711S), following the manufacturer's recommendations. The intact deglycosylated sample was analyzed by reverse-phase liquid chromatography on a BioAccord system using a Protein BEH C4 300Å Waters (Cat. nr. 186004495). The data was processed using MaxEnt1 software.

### Results and Discussion

### Lead IgG Generation and Screening

Anti-CD203c antibodies carrying the anti-c-kit MH1 light chain (described in Table 2) were readily expressed and purified from CHO cells using Protein-A affinity chromatography. Analytical SEC demonstrated that all clones were >95% monomeric and preparations had a low endotoxin content <1 EU/mg.

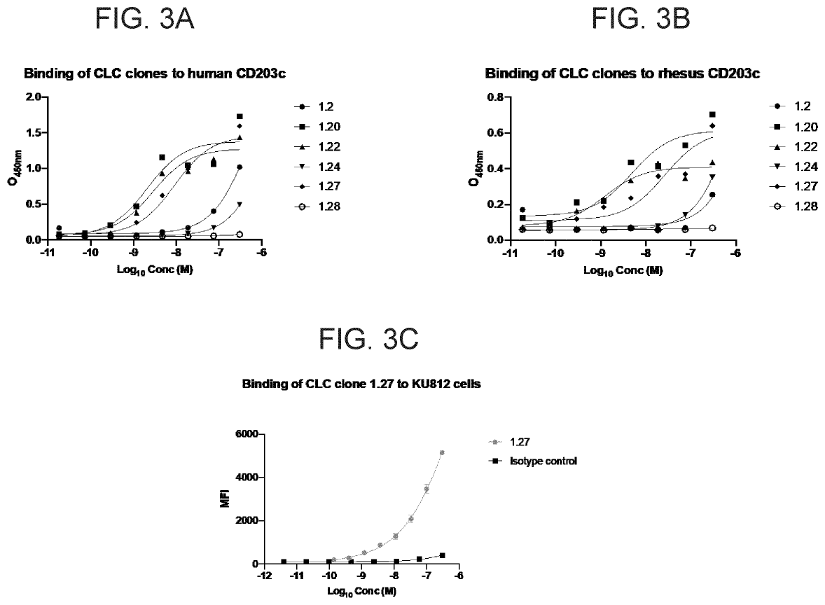

Purified IgGs were initially tested for binding to His-tagged recombinant versions of human and rhesus CD203c (FIG. 3A and FIG. 3B) and all clones that demonstrated measurable binding to both orthologs were brought forward for binding analysis using flow cytometry on KU812 cells, which endogenously express human CD203c. Of the clones tested, only 1.27 retained robust binding to cell-surface expressed CD203c (FIG. 3C) and this was selected as the parental clone for subsequent heavy chain mutagenesis and variant library generation.

### 1.27 Heavy Chain Library Generation and Screening

Anti-CD203c parental clone 1.27 was used as a template for generation of two heavy chain-precision variant libraries which are described in FIG. 4 and FIG. 5. These libraries did not target the light chain, with all variants maintaining the common anti-c-kit MH1 light chain described in Table 2. The libraries were selected over 4 iterative rounds of phage selections on human and rhesus CD203c and screened in periprep Fab format for the ability to bind to both orthologs by direct binding ELISA. Subsequent sequence analysis allowed the selection of a lead panel of optimized clones with sequences derived from both HCDR1 and HCDR2 and HCDR3 libraries.

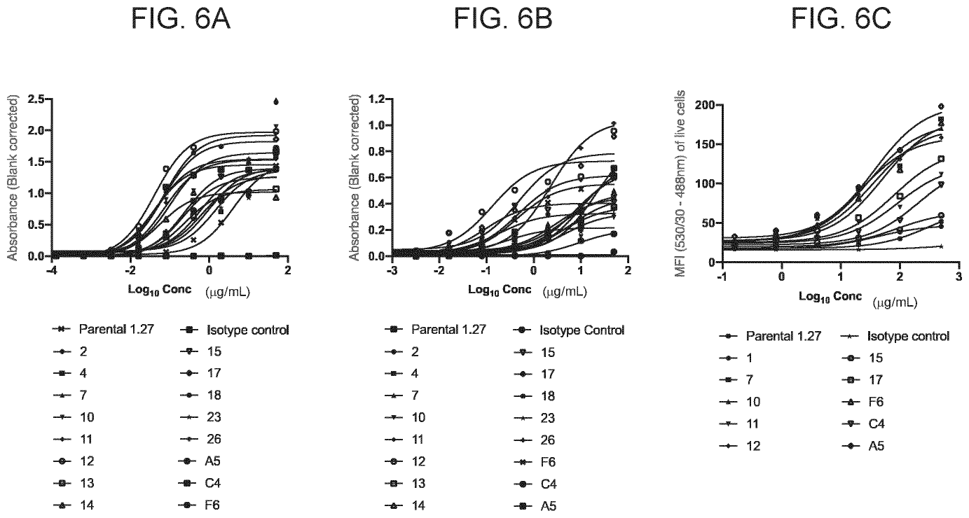

The 1.27 output clones summarized in Table 3 were expressed and purified as IgGs and re-tested in purified IgG format for human/rhesus CD203c binding by direct ELISA, and for binding to cell surface CD203c on KU812 cells using a flow-based assay. Based on the data summarized in FIG. 6, the top performing clones were prioritized for common light chain bispecific generation. Clones generated included HCDR1/HCDR2 variants, HCDR3 variants and clones that combined mutations derived from both libraries, these are summarized in Table 3.

### Characterization of Optimized Common Light Chain Bispecifics

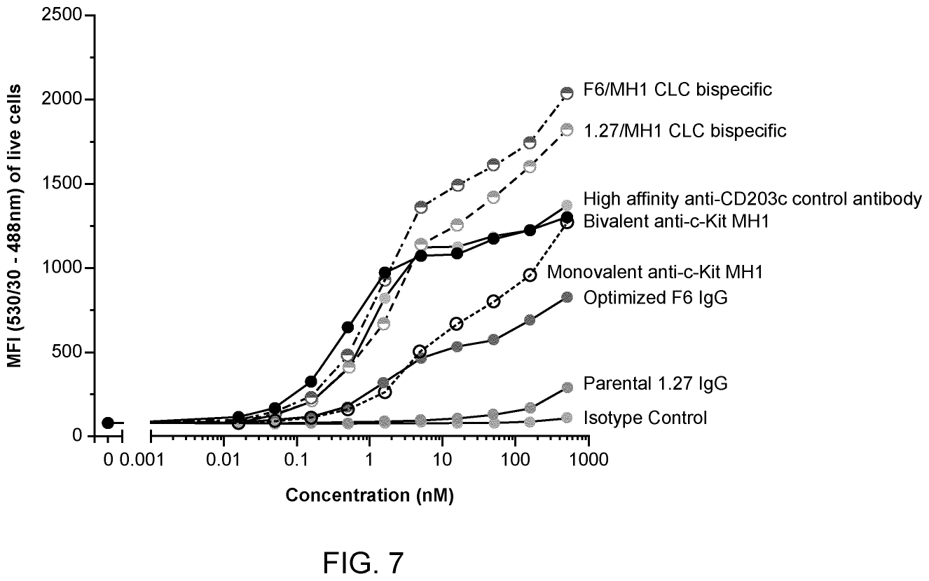

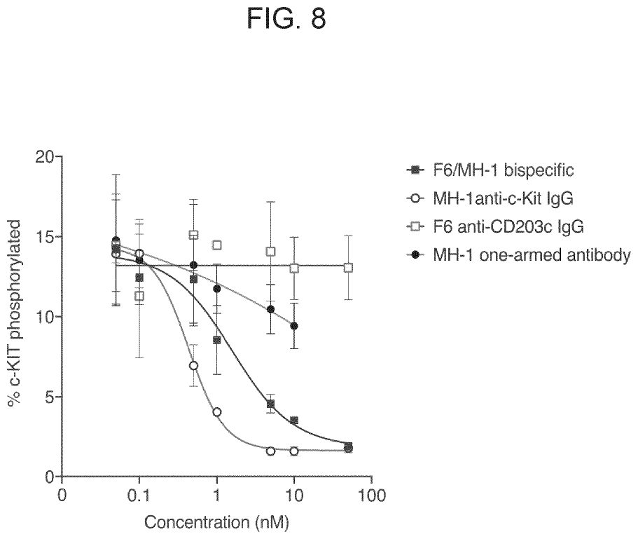

Common light chain bispecific molecules produced from heavy and light chain sequences detailed in Tables 1-3 were expressed in CHO cells and purified by Protein-A affinity chromatography followed by a preparative cation-exchange chromatography step to achieve >95% monomeric fraction. The purified proteins were assessed for co-operative binding to both targets, CD203c and c-Kit, on KU812 cells. FIG. 7 demonstrates that bispecific molecules engaging dual receptors on KU812 cells, 1.27/MH1 & F6/MH1, have enhanced binding when compared to bivalent CD203c IgGs and bivalent and monovalent c-Kit IgGs. This enhanced binding translates to potent inhibition of c-Kit receptor phosphorylation by the F6/MH1 bispecific molecule when compared to monovalent anti-c-kit, which is shown in FIG. 8. This inhibition demonstrates that monovalent c-Kit targeting can inhibit receptor phosphorylation, and co-engagement with CD203c greatly enhances the potency of this inhibition through enhanced receptor occupancy.

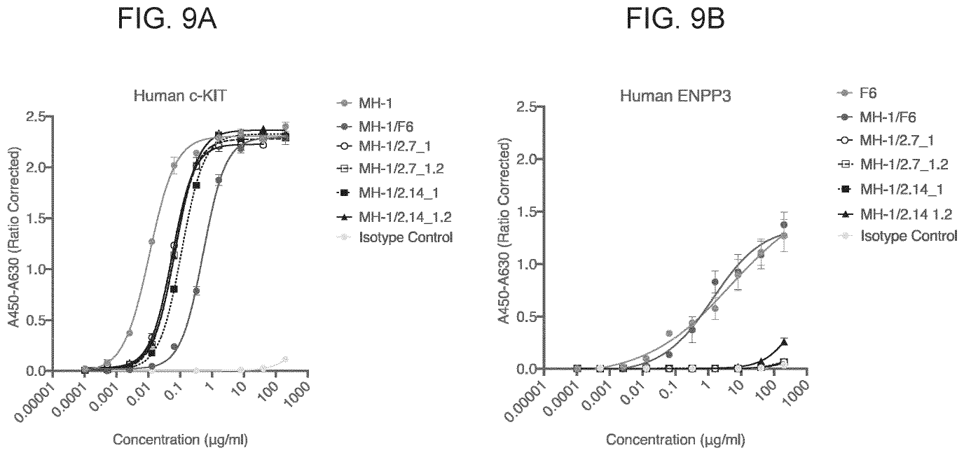

However, not all bispecific pairs retained binding to both targets. When alternative common light chains derived from anti-CD203c binding domains were grafted with MH1CDRs (Table 2; SEQ ID NOs: 58-61) and paired with CD203c VH domains (Table 3; SEQ ID NOs: 54-57), c-Kit binding was retained but binding to CD203c was lost. These data are summarized in FIG. 9A and FIG. 9B, demonstrating that successful bispecific pairs could not be predicted a priori.

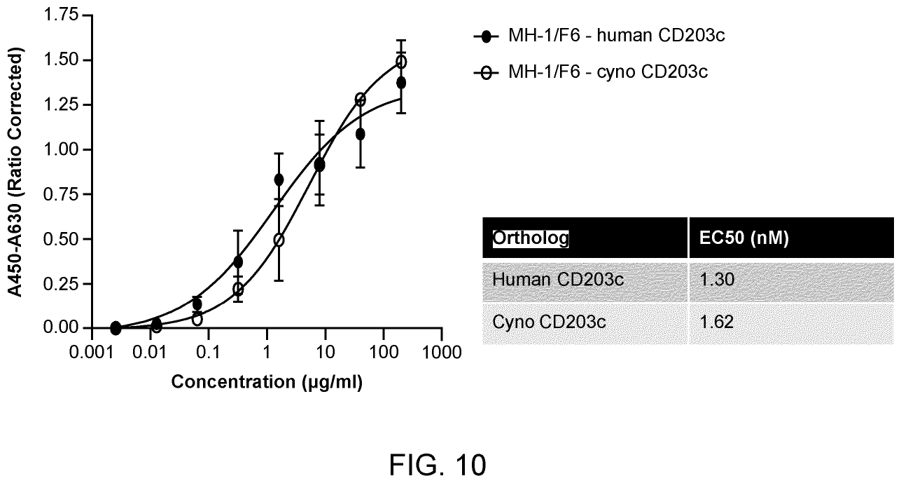

Comparative binding of the F6/MH1 bispecific molecule to human and cynomolgus CD203c was tested by ELISA. Data shown in FIG. 10 demonstrate equivalent binding to both orthologs.

### Affinity Optimization of Anti-CD203c 1.27 Binding Domain

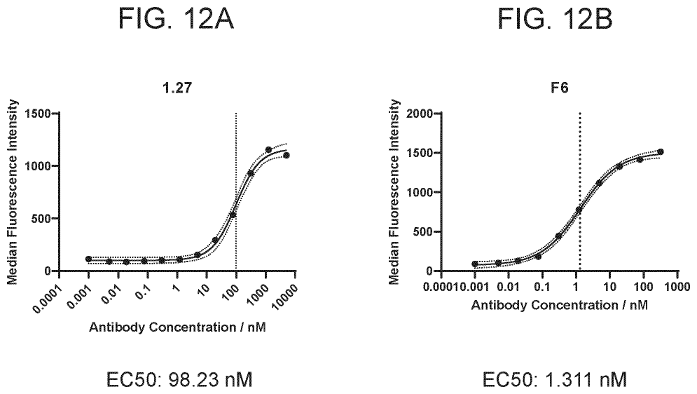

SPR was used to characterize variants from the 1.27 affinity optimization library. Binding of Fab versions of the parental 1.27, F6 with mutations in H-CDR1 & H-CDR2 and F6.12 with mutations across all three VH CDRs was measured to chips coated with biotinylated human or cyno CD203c. 1:1 interaction kinetic determinations demonstrated an 10-fold improvement in KD for F6 at 74 nM, with a further improvement to 55 nM for F6.12 (FIG. 11A-FIG. 11D). This improvement in binding was further demonstrated on KU812 cells with a 75-fold improvement in binding for F6 IgG (EC50 1.311 nM) compared to parental 1.27 IgG (EC50 98.23 nM). This is summarized in FIG. 12A-FIG. 12B.

### pI Engineering of MH1/F6.12 Bispecific

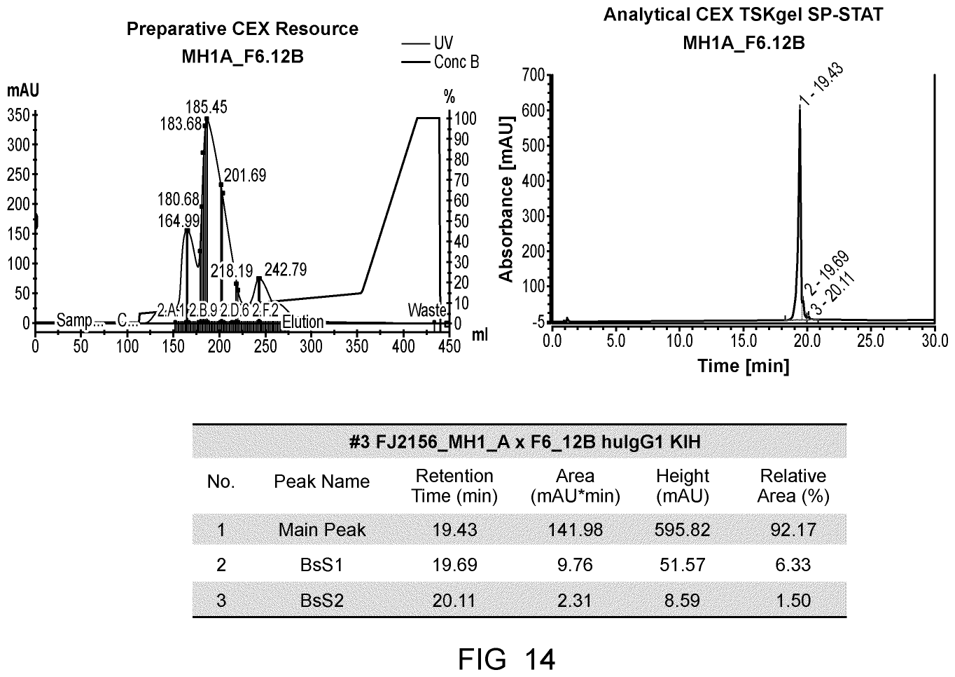

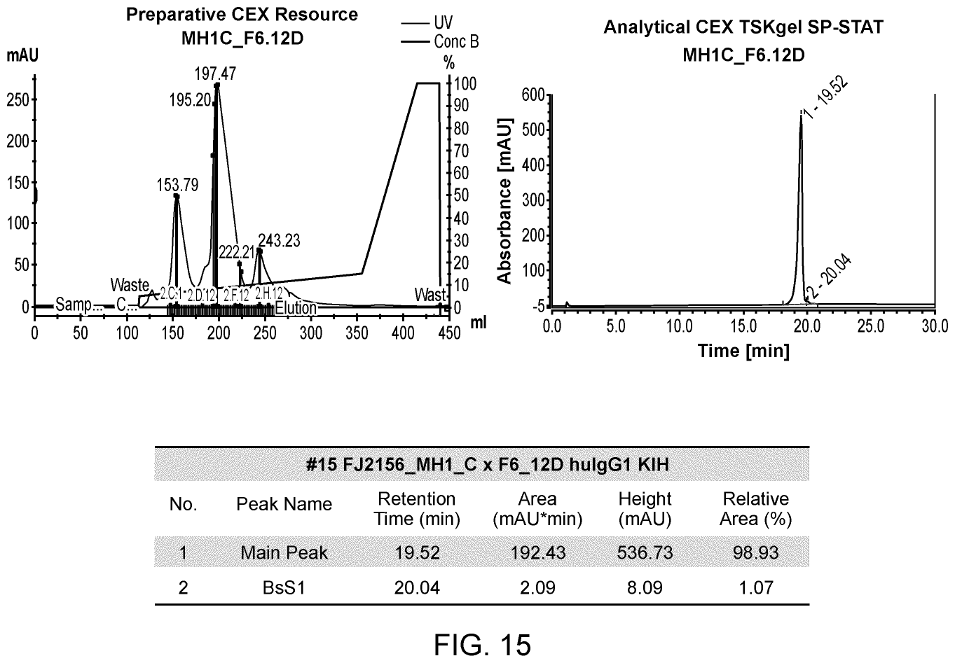

pI engineering of both of the bispecific heavy chains was undertaken to improve downstream process development and optimize heterodimer formation. 5 double mutants were generated for the anti-c-Kit VH MH1 to drive towards a more negative pI, with 5 double mutants of the anti-CD203c VH F6.12 driving for a more positive pI. These mutations are summarized in FIG. 13. A matrix of all possible 25 variant bispecifics was generated and compared for productivity, analytical SEC and pilot heterodimer formation by analytical CEX. Each bispecific variant differed in CEX profile with some showing multiple peaks that were not easily resolved. An example of such a profile is shown in FIG. 14 where 3 overlapping peaks are present for MH1_A/F6.12_B. However, some variants showed clear resolution and preferential heterodimer formation which could be purified by preparative CEX with robust yields. This was the case for MH1_C/F6.12_D which is described in FIG. 15.

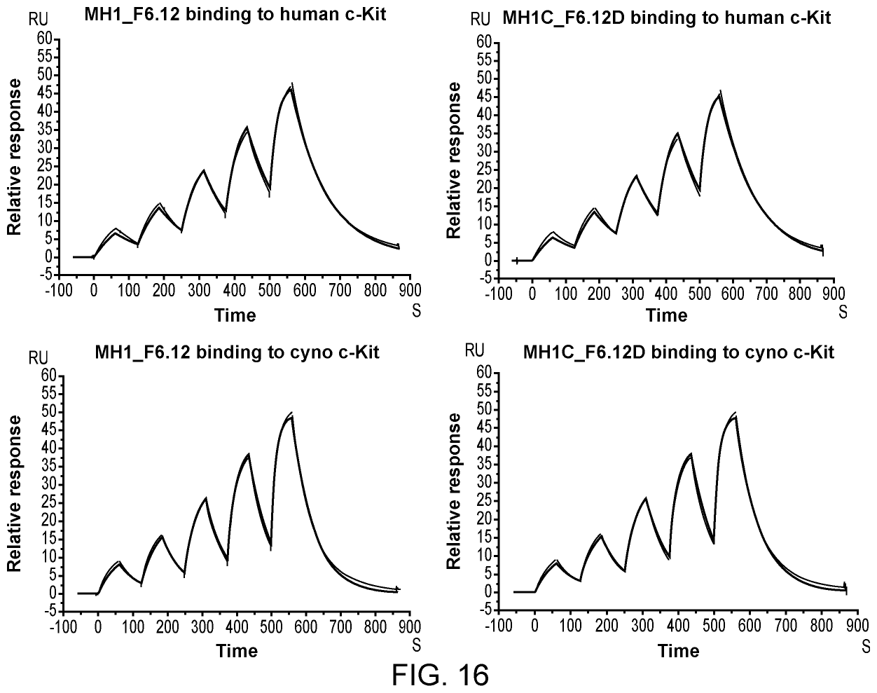

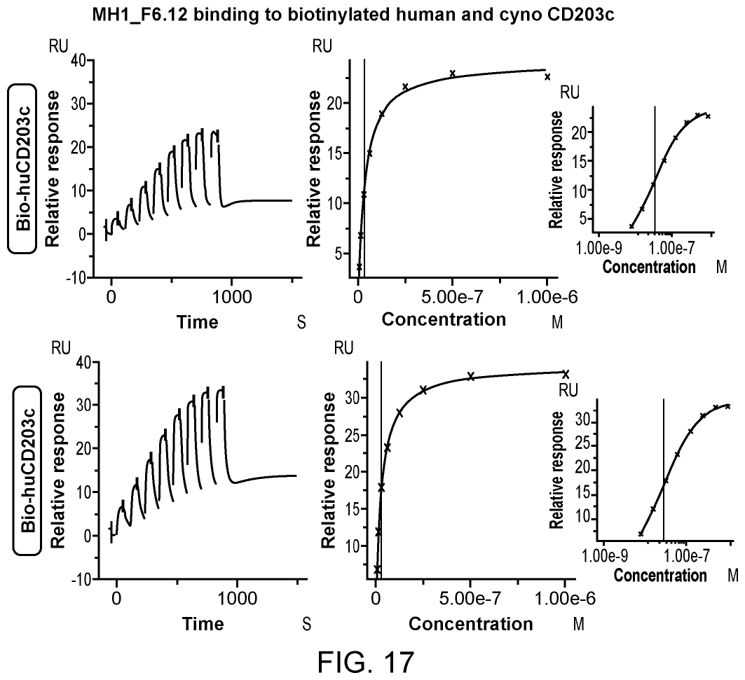

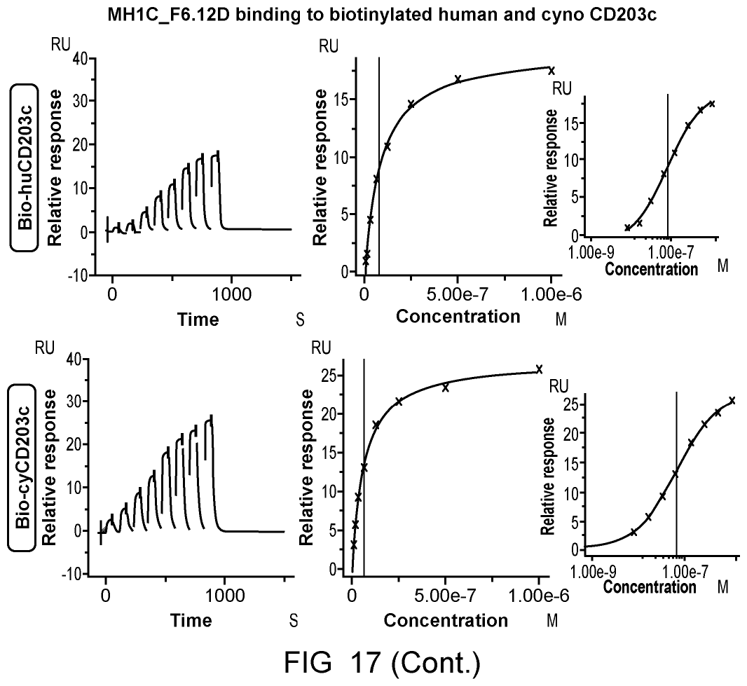

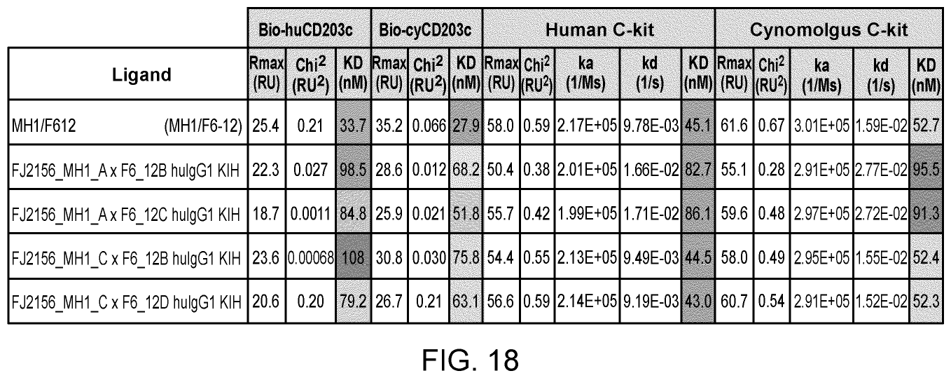

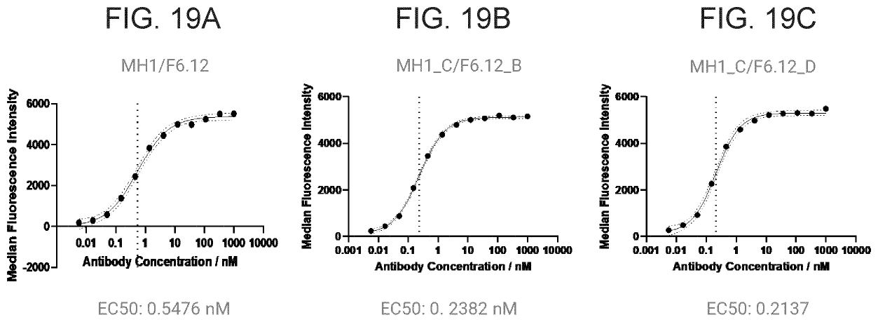

MH1_C/F6.12_D was compared to the parental bispecific MH1/F6.12 to ensure that interaction with dual targets was maintained post-pI engineering. Both variants demonstrated equivalent binding to human and cyno c-Kit (FIG. 16) and to human and cyno CD203c (FIG. 17) with kinetic data summarized in FIG. 18. This was also true in terms of binding to cell surface targets with equivalent binding demonstrated pre and post-pI engineering on KU812 cells (FIG. 19A-FIG. 19C).

1. A bispecific protein comprising:
(a) a first polypeptide chain comprising a first immunoglobulin heavy chain comprising an anti-c-Kit heavy chain variable (VH) domain;
(b) a second polypeptide chain comprising a second immunoglobulin heavy chain comprising an anti-CD203c VH domain; and
(c) identical third and fourth polypeptide chains, wherein the third polypeptide chains and the fourth polypeptide chain each comprise an immunoglobulin light chain comprising a light chain variable (VL) domain that is capable of specifically binding c-Kit and CD203c.

2. The bispecific protein of claim 1, wherein
the anti-c-Kit VH domain comprises a heavy chain complementarity determining region 1 (HCDR1) comprising the amino acid sequence of SEQ ID NO: 2, a heavy chain complementarity determining region 2 (HCDR2) comprising the amino acid sequence of SEQ ID NO: 3, and a heavy chain complementarity determining region 3 (HCDR3) comprising the amino acid sequence of SEQ ID NO: 4;
the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 73; and
the VL domain comprises a light chain complementarity determining region 1 (LCDR1) comprising the amino acid sequence of SEQ ID NO: 6, a light chain complementarity determining region 2 (LCDR2) comprising the amino acid sequence of SEQ ID NO: 7, and a light chain complementarity determining region 3 (LCDR3) comprising the amino acid sequence of SEQ ID NO: 8.

3. The bispecific protein of claim 1, wherein
the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4;
the anti-c-CD203c VH domain comprises a HCDR1 comprising the amino acid sequence of SEQ ID NO: 10, a HCDR2 comprising the amino acid sequence of SEQ ID NO: 11, and a HCDR3 comprising the amino acid sequence of SEQ ID NO: 12; and
the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

4. The bispecific protein of claim 1, wherein
the anti-c-Kit VH domain comprises a HCDR1 comprising the amino acid sequence of SEQ ID NO: 2, a HCDR2 comprising the amino acid sequence of SEQ ID NO: 3, and a HCDR3 comprising the amino acid sequence of SEQ ID NO: 4;
the anti-c-CD203c VH domain comprises a HCDR1 comprising the amino acid sequence of SEQ ID NO: 14, a HCDR2 comprising the amino acid sequence of SEQ ID NO: 15, and a HCDR3 comprising the amino acid sequence of SEQ ID NO: 12; and
the VL domain comprises a LCDR1 comprising the amino acid sequence of SEQ ID NO: 6, a LCDR2 comprising the amino acid sequence of SEQ ID NO: 7, and a LCDR3 comprising the amino acid sequence of SEQ ID NO: 8.

5. The bispecific protein of claim 2, wherein
the anti-c-Kit VH domain comprises the amino acid sequence of SEQ ID NO: 65;
the anti-c-CD203c VH domain comprises the amino acid sequence of SEQ ID NO: 71; and
the VL domain comprises the amino acid sequence of SEQ ID NO: 5.

6. The bispecific protein of claim 3, wherein
the anti-c-Kit VH domain comprises the amino acid sequence of SEQ ID NO: 1;
the anti-c-CD203c VH domain comprises the amino acid sequence of SEQ ID NO: 9; and
the VL domain comprises the amino acid sequence of SEQ ID NO: 5.

7. The bispecific protein of claim 4, wherein
the anti-c-Kit VH domain comprises the amino acid sequence of SEQ ID NO: 1;
the anti-c-CD203c VH domain comprises the amino acid sequence of SEQ ID NO: 13; and
the VL domain comprises the amino acid sequence of SEQ ID NO: 5.

8. The bispecific protein of claim 1, wherein
the anti-c-Kit VH domain comprises the amino acid sequence of SEQ ID NO: 1;
the anti-c-CD203c VH domain comprises the amino acid sequence of any one of SEQ ID NOs: 16-51; and
the VL domain comprises the amino acid sequence of SEQ ID NO: 5.

9. The bispecific protein of claim 1, wherein
the anti-c-Kit VH domain comprises the amino acid sequence of any one of SEQ ID NOs: 63-67;
the anti-c-CD203c VH domain comprises the amino acid sequence of any one of SEQ ID NOs: 68-72; and
the VL domain comprises the amino acid sequence of SEQ ID NO: 5.

10. The bispecific protein of any one of claims 1-9, wherein the bispecific protein comprises an immunoglobulin constant region.

11. The bispecific protein of claim 10, wherein the immunoglobulin constant region is an IgG, IgE, IgM, IgD, IgA or IgY immunoglobulin constant region.

12. The bispecific protein of claim 10, wherein the immunoglobulin constant region is an IgG1, IgG2, IgG3, IgG4, IgA1 or IgA2 immunoglobulin constant region.

13. The bispecific protein of claim 10, wherein the immunoglobulin constant region is an immunologically inert constant region.

14. The bispecific protein of any one of claims 1-9, wherein the first polypeptide chain comprises a first immunoglobulin constant region and the second polypeptide chain comprises a second immunoglobulin constant region, and wherein the first immunoglobulin constant region and the second immunoglobulin constant region comprise knob-in-hole mutations.

15. The bispecific protein of claim 14, wherein the first immunoglobulin constant region comprises a CH3 domain comprising the amino acid substitutions S354C and T366W, and wherein the second immunoglobulin constant region comprises a CH3 domain comprising the amino acid substitutions Y349C, T366S, L368A, and Y407V, wherein numbering is according to the EU index as in Kabat.

16. The bispecific protein of claim 14, wherein the first immunoglobulin constant region comprises a CH3 domain comprising the amino acid substitutions Y349C, T366S, L368A, and Y407V, and wherein the second immunoglobulin constant region comprises a CH3 domain comprising the amino acid substitutions S354C and T366W, wherein numbering is according to the EU index as in Kabat.

17. The bispecific protein of claim 14, wherein
(a) the first immunoglobulin constant region comprises the amino acid sequence of SEQ ID NO: 52, and the second immunoglobulin constant region comprises the amino acid sequence of SEQ ID NO: 53; or
(b) the first immunoglobulin constant region comprises the amino acid sequence of SEQ ID NO: 53, and the second immunoglobulin constant region comprises the amino acid sequence of SEQ ID NO: 52.

18. A bispecific protein that binds c-Kit and CD203c, wherein the c-Kit and CD203c are on the surface of the same cell.

19. The bispecific protein of claim 18, wherein the bispecific protein is a designed ankyrin repeat protein (DARPin), a tandem VHH, or a tandem immunoglobulin new antigen receptor (IgNAR).

20. An immunoconjugate comprising the bispecific protein of any one of claims 1-19, linked to a therapeutic agent.

21. The immunoconjugate of claim 20, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

22. A pharmaceutical composition comprising the bispecific protein of any one of claims 1-19 or the immunoconjugate of claim 20 or 21, and a pharmaceutically acceptable carrier, diluent or excipient.

23. A nucleic acid molecule encoding the first polypeptide chain, the second polypeptide chain, the third polypeptide chain, and the fourth polypeptide chain of the bispecific protein of any one of claims 1-17.

24. A nucleic acid molecule encoding the bispecific protein of claim 18 or 19.

25. An expression vector comprising the nucleic acid molecule of claim 22 or 23.

26. A recombinant host cell comprising the nucleic acid molecule of claim 23 or 24 or the expression vector of claim 25.

27. A method of producing a bispecific protein, the method comprising:
culturing the recombinant host cell of claim 26 under conditions whereby the nucleic acid molecule is expressed, thereby producing the protein; and
isolating the protein from the host cell or culture.

28. A method for treating an inflammatory disease or a neoplasm in a subject in need thereof, the method comprising administering to the subject a therapeutically effective amount of the bispecific protein of any one of claims 1-19, the immunoconjugate of claim 20 or 21, or the pharmaceutical composition of claim 22.

29. A method for ameliorating a symptom of an inflammatory disease or a neoplasm in a subject in need thereof, the method comprising administering to the subject a therapeutically effective amount of the bispecific protein of any one of claims 1-19, the immunoconjugate of claim 20 or 21, or the pharmaceutical composition of claim 22.

30. The method of claim 28 or 29, wherein the inflammatory disease is a chronic inflammatory disease.

31. The method of claim 28 or 29, wherein the inflammatory disease is a mast cell-driven disease.

32. The method of claim 28 or 29, wherein the inflammatory disease is chronic urticaria, chronic pruritus, atopic dermatitis, allergic asthma, prurigo nodularis, eosinophilic gastritis, eosinophilic duodenitis, or eosinophilic esophagitis.

33. The method of claim 28 or 29, wherein the neoplasm is a mast cell-driven neoplasm.

34. The method of claim 28 or 29, wherein the neoplasm is systemic mastocytosis or mast cell leukemia.

35. The bispecific protein of any one of claims 1-19, the immunoconjugate of claim 20 or 21, or the pharmaceutical composition of claim 22 for use as a medicament.

36. The bispecific protein of any one of claims 1-19, the immunoconjugate of claim 20 or 21, or the pharmaceutical composition of claim 22 for use in the treatment of an inflammatory disease or a neoplasm.

37. The bispecific protein, the immunoconjugate, or the pharmaceutical composition for use of claim 36, wherein the inflammatory disease is a chronic inflammatory disease.

38. The bispecific protein, the immunoconjugate, or the pharmaceutical composition for use of claim 36, wherein the inflammatory disease is a mast cell-driven disease.

39. The bispecific protein, the immunoconjugate, or the pharmaceutical composition for use of claim 36, wherein the inflammatory disease is chronic urticaria, chronic pruritus, atopic dermatitis, allergic asthma, prurigo nodularis, eosinophilic gastritis, eosinophilic duodenitis, or eosinophilic esophagitis.

40. The bispecific protein, the immunoconjugate, or the pharmaceutical composition for use of claim 36, wherein the neoplasm is a mast cell-driven neoplasm.

41. The bispecific protein, the immunoconjugate, or the pharmaceutical composition for use of claim 36, wherein the neoplasm is systemic mastocytosis or mast cell leukemia.


SEQUENCE LISTING (73 sequences)

SEQ ID NO: 1 - protein, 116 aa, synthetic construct
QVQLVQSGAEVKKPGASVKVSCKASGYTFTDYYMNWVRQAPGQGLEWMGRIYPGSGNTYYAQKFQGRVTMTRDTSTSTVYMELSSLRSEDTAVYYCARGVYYYDYWGQGTLVTVSS

SEQ ID NO: 2 - protein, 10 aa, synthetic construct
GYTFTDYYMN

SEQ ID NO: 3 - protein, 12 aa, synthetic construct
RIYPGSGNTYYA

SEQ ID NO: 4 - protein, 9 aa, synthetic construct
ARGVYYYDY

SEQ ID NO: 5 - protein, 107 aa, synthetic construct
DIQMTQSPSSLSASVGDRVTITCRASQGIRTNLAWFQQKPGKAPKSLIYAASSLQSGVPSRFSGSGSGTDFTLTISSLQPEDFATYYCQQYNSYPRTFGGGTKVEIK

SEQ ID NO: 6 - protein, 11 aa, synthetic construct
RASQGIRTNLA

SEQ ID NO: 7 - protein, 11 aa, synthetic construct
AASSLQSGVPS

SEQ ID NO: 8 - protein, 9 aa, synthetic construct
QQYNSYPRT

SEQ ID NO: 9 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYYAMDVWGQGTTVTVSS

SEQ ID NO: 10 - protein, 10 aa, synthetic construct
GYRFTSYWIG

SEQ ID NO: 11 - protein, 12 aa, synthetic construct
IIYPGDSDTRYS

SEQ ID NO: 12 - protein, 12 aa, synthetic construct
ARKDYYYYAMDV

SEQ ID NO: 13 - protein, 119 aa, synthetic construct
EVQLVQSGAEVKKPGESLKISCKGSGYRFASNWIGWVRQMPGKGLEWMGEIYPGDSNTRYSPSFQGQVTISADKSISTAYLQWSSLKASDTAMYYCARKDYYYYAMDVWGQGTTVTVSS

SEQ ID NO: 14 - protein, 10 aa, synthetic construct
GYRFASNWIG

SEQ ID NO: 15 - protein, 12 aa, synthetic construct
EIYPGDSNTRYS

SEQ ID NO: 16 - protein, 119 aa, synthetic construct
EVQLVQSGAEVKKPGESLKISCKGSGYRFGSYWIGWVRQMPGKGLEWMGDIYPGNSDTRYSPSFQGQVTISADKSISTAYLQWSSLKASDTAMYYCARKDYYYYAMDVWGQGTTVTVSS

SEQ ID NO: 17 - protein, 119 aa, synthetic construct
EVQLVQSGAEVKKPGESLKISCKGSGYRFSSYRIGWVRQMPGKGLEWMGEIYPGDSKTRYSPSFQGQVTISADKSISTAYLQWSSLKASDTAMYYCARKDYYYYAMDVWGQGTTVTVSS

SEQ ID NO: 18 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARHDYYYGAMDVWGQGTTVTVSS

SEQ ID NO: 19 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARIDYYHYAMDVWGQGTTVTVSS

SEQ ID NO: 20 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARQDYYYGAMDVWGQGTTVTVSS

SEQ ID NO: 21 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARADEYYYAMDVWGQGTTVTVSS

SEQ ID NO: 22 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARADYLYYAMDVWGQGTTVTVSS

SEQ ID NO: 23 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARNDYYYGAMDVWGQGTTVTVSS

SEQ ID NO: 24 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCAREDYYYQAMDVWGQGTTVTVSS

SEQ ID NO: 25 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARFDIYYYAMDVWGQGTTVTVSS

SEQ ID NO: 26 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGAMDVWGQGTTVTVSS

SEQ ID NO: 27 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGAFDVWGQGTTVTVSS

SEQ ID NO: 28 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGIMDVWGQGTTVTVSS

SEQ ID NO: 29 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGSMDVWGQGTTVTVSS

SEQ ID NO: 30 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGALDVWGQGTTVTVSS

SEQ ID NO: 31 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYGTMDVWGQGTTVTVSS

SEQ ID NO: 32 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYEGAMDVWGQGTTVTVSS

SEQ ID NO: 33 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYNAMPVWGQGTTVTVSS

SEQ ID NO: 34 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYDAMDYWGQGTTVTVSS

SEQ ID NO: 35 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYTYYAMDVWGQGTTVTVSS

SEQ ID NO: 36 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDYYYSADDVWGQGTTVTVSS

SEQ ID NO: 37 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDEYYAAMDVWGQGTTVTVSS

SEQ ID NO: 38 - protein, 119 aa, synthetic construct
EVQLVQSGAEVKKPGESLKISCKGSGYRFTSYWIGWVRQMPGKGLEWMGIIYPGDSDTRYSPSFQGQVTISADKSISTAYLQWSSLKASDTAMYYCARKDEYDYAMDVWGQGTTVTVSS

SEQ ID NO: 39 - protein, 119 aa, synthetic construct
EVQLVQSGAEVKKPGESLKISCKGSGYRFGSYWIGWVRQMPGKGLEWMGEIYPGDSKTRYSPSFQGQVTISADKSISTAYLQWSSLKASDTAMYYCARKDYYYYAMDVWGQGTTVTVSS

SEQ ID NO: 40 - protein, 119 aa, synthetic construct
EVQLVQSGAEVKKPGESLKISCKGSGYRFRSYWIGWVRQMPGKGLEWMGIIYSGDSDTKYSPSFQGQVTISADKSISTAYLQWSSLKASDTAMYYCARKDYYYYAMDVWGQGTTVTVSS

SEQ ID NO: 41 - protein, 119 aa, synthetic construct
EVQLVQSGAEVKKPGESLKISCKGSGYRFASTWIGWVRQMPGKGLEWMGIIYPGDSQTRYRPSFQGQVTISADKSISTAYLQWSSLKASDTAMYYCARKDYYYYAMDVWGQGTTVTVSS

SEQ ID NO: 42 - protein, 119 aa, synthetic construct
EVQLVQSGAEVKKPGESLKISCKGSGYRFASHWIGWVRQMPGKGLEWMGEIYPGDSDTRISPSFQGQVTISADKSISTAYLQWSSLKASDTAMYYCARKDYYYYAMDVWGQGTTVTVSS

SEQ ID NO: 43 - protein, 119 aa, synthetic construct
EVQLVQSGAEVKKPGESLKISCKGSGYTFTSYFIGWVRQMPGKGLEWMGIIYPGDSDSRRSPSFQGQVTISADKSISTAYLQWSSLKASDTAMYYCARKDYYYYAMDVWGQGTTVTVSS

SEQ ID NO: 44 - protein, 119 aa, synthetic construct
EVQLVQSGAEVKKPGESLKISCKGSGVRFTSYDIGWVRQMPGKGLEWMGIIYTGDSDTRVSPSFQGQVTISADKSISTAYLQWSSLKASDTAMYYCARKDYYYYAMDVWGQGTTVTVSS

SEQ ID NO: 45 - protein, 119 aa, synthetic construct
EVQLVQSGAEVKKPGESLKISCKGSGYRFTSEWIGWVRQMPGKGLEWMGIIYTGDSDTKYSPSFQGQVTISADKSISTAYLQWSSLKASDTAMYYCARKDYYYYAMDVWGQGTTVTVSS

SEQ ID NO: 46 - protein, 119 aa, synthetic construct
EVQLVQSGAEVKKPGESLKISCKGSGYRFISYWIGWVRQMPGKGLEWMGIIWPGDSDTRYQPSFQGQVTISADKSISTAYLQWSSLKASDTAMYYCARKDYYYYAMDVWGQGTTVTVSS

SEQ ID NO: 47 - protein, 119 aa, synthetic construct
EVQLVQSGAEVKKPGESLKISCKGSGSRFTSYWIRWVRQMPGKGLEWMGIIYPGDSDTRFGPSFQGQVTISADKSISTAYLQWSSLKASDTAMYYCARKDYYYYAMDVWGQGTTVTVSS

SEQ ID NO: 48 - protein, 119 aa, synthetic construct
EVQLVQSGAEVKKPGESLKISCKGSGYKFTSDWIGWVRQMPGKGLEWMGIINPGDSDTRYSPSFQGQVTISADKSISTAYLQWSSLKASDTAMYYCARKDYYYYAMDVWGQGTTVTVSS

SEQ ID NO: 49 - protein, 119 aa, synthetic construct
EVQLVQSGAEVKKPGESLKISCKGSGYRFASNWIGWVRQMPGKGLEWMGEIYPGDSNTRYSPSFQGQVTISADKSISTAYLQWSSLKASDTAMYYCARNDYYYGAMDVWGQGTTVTVSS

SEQ ID NO: 50 - protein, 119 aa, synthetic construct
EVQLVQSGAEVKKPGESLKISCKGSGYRFASNWIGWVRQMPGKGLEWMGEIYPGDSNTRYSPSFQGQVTISADKSISTAYLQWSSLKASDTAMYYCARKDYYYGAMDVWGQGTTVTVSS

SEQ ID NO: 51 - protein, 119 aa, synthetic construct
EVQLVQSGAEVKKPGESLKISCKGSGYRFASNWIGWVRQMPGKGLEWMGEIYPGDSNTRYSPSFQGQVTISADKSISTAYLQWSSLKASDTAMYYCARKDYYYGIMDVWGQGTTVTVSS

SEQ ID NO: 52 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 121 aa, synthetic construct
EVQLVESGPGLVKPSETLSLTCTVSGGSISSSSYYWGWIRQPPGKGLEWIGSMYYSGSTYHNPSLKSRVIISVDTSKNQFSLKLSSVTAADTAVYYCARHYITVAGIFDYWGQGTLVTVSS

SEQ ID NO: 55 - protein, 121 aa, synthetic construct; identical to SEQ ID NO: 54

SEQ ID NO: 56 - protein, 123 aa, synthetic construct
QVQLQESGPGLVKPSQTLSLTCTVSGGSISSGGYYWSWIRQHPGKGLEWIGIIYYSGSTYYNPSLKSRVTISVDTSKNQFSLKLNSVTAADTAVFYCARVAIVTTIPGGMDVWGQGTTVTVSS

SEQ ID NO: 57 - protein, 123 aa, synthetic construct; identical to SEQ ID NO: 56

SEQ ID NO: 58 - protein, 106 aa, synthetic construct
QSVLTQPPSASGTPGQRATISCRASQGIRTNLAWYQQVPGTAPKLLIYAASSLQSGVPSRFSGSKSGTSASLAISGLQSEDEADYYCQQYNSYPRTFGGGTKLTVL

SEQ ID NO: 59 - protein, 106 aa, synthetic construct
QSVLTQPPSASGTPGQRATISCRASQGIRTNLAWYQQVPGTAPKLLIYSNNQRPSGVPDRFSGSKSGTSASLAISGLQSEDEADYYCQQYNSYPRTFGGGTKLTVL

SEQ ID NO: 60 - protein, 107 aa, synthetic construct
EIVLTQSPDFQSVTPKEKVTITCRASQGIRTNLAWYQQKPDQSPKLLIKAASSLQSGVPSRFSGSGSGTDFTLTINSLEAEDAATYYCQQYNSYPRTFGQGTKVEIK

SEQ ID NO: 61 - protein, 107 aa, synthetic construct
EIVLTQSPDFQSVTPKEKVTITCRASQGIRTNLAWYQQKPDQSPKLLIKYASQSFSGVPSRFSGSGSGTDFTLTINSLEAEDAATYYCQQYNSYPRTFGQGTKVEIK

SEQ ID NO: 62 - protein, 107 aa, Homo sapiens [sequence omitted]

SEQ ID NO: 63 - protein, 116 aa, synthetic construct
EVQLVQSGAEVKKPGASVKVSCKASGYTFTDYYMNWVRQAPGQGLEWMGRIYPGSGNTYYAQKFQDRVTMTRDTSTSTVYMELSSLRSEDTAVYYCARGVYYYDYWGQGTLVTVSS

SEQ ID NO: 64 - protein, 116 aa, synthetic construct
QVQLVQSGAEVKEPGASVKVSCKASGYTFTDYYMNWVRQAPGQGLEWMGRIYPGSGNTYYAEKFQGRVTMTRDTSTSTVYMELSSLRSEDTAVYYCARGVYYYDYWGQGTLVTVSS

SEQ ID NO: 65 - protein, 116 aa, synthetic construct
QVQLVQSGAEVKEPGASVKVSCKASGYTFTDYYMNWVRQAPGQGLEWMGRIYPGSGNTYYAQNFQGRVTMTRDTSTSTVYMELSSLRSEDTAVYYCARGVYYYDYWGQGTLVTVSS

SEQ ID NO: 66 - protein, 116 aa, synthetic construct
QVQLVQSGAEVKEPGASVKVSCKASGYTFTDYYMNWVRQAPGQGLEWMGRIYPGSGNTYYAQKFEGRVTMTRDTSTSTVYMELSSLRSEDTAVYYCARGVYYYDYWGQGTLVTVSS

SEQ ID NO: 67 - protein, 116 aa, synthetic construct
QVQLVQSGAEVKEPGASVKVSCKASGYTFTDYYMNWVRQAPGQGLEWMGRIYPGSGNTYYAQKFQDRVTMTRDTSTSTVYMELSSLRSEDTAVYYCARGVYYYDYWGQGTLVTVSS

SEQ ID NO: 68 - protein, 119 aa, synthetic construct
QVQLVQSGAEVKKPGGSLKISCKGSGYRFASNWIGWVRQMPGKGLEWMGEIYPGDSNTRYSPSFQGQVTISADKSISTAYLQWSSLKASDTAMYYCARKDYYYGIMDVWGQGTTVTVSS

SEQ ID NO: 69 - protein, 119 aa, synthetic construct
QVQLVQSGAEVKKPGESLKISCKGSGYRFASNWIGWVRQMPGKGLQWMGEIYPGDSNTRYSPSFQGQVTISADKSISTAYLQWSSLKASDTAMYYCARKDYYYGIMDVWGQGTTVTVSS

SEQ ID NO: 70 - protein, 119 aa, synthetic construct
QVQLVQSGAEVKKPGESLKISCKGSGYRFASNWIGWVRQMPGKGLEWMGEIYPGDSNTRYRPSFQGQVTISADKSISTAYLQWSSLKASDTAMYYCARKDYYYGIMDVWGQGTTVTVSS

SEQ ID NO: 71 - protein, 119 aa, synthetic construct
QVQLVQSGAEVKKPGESLKISCKGSGYRFASNWIGWVRQMPGKGLEWMGEIYPGDSNTRYSPSFQGQVTISADKSISTAYLQWSRLKASDTAMYYCARKDYYYGIMDVWGQGTTVTVSS

SEQ ID NO: 72 - protein, 119 aa, synthetic construct
QVQLVQSGAEVKKPGESLKISCKGSGYRFASNWIGWVRQMPGKGLEWMGEIYPGDSNTRYSPSFQGQVTISADKSISTAYLQWSSLKASDTAMYYCARKDYYYGIMDVWGKGTTVTVSS

SEQ ID NO: 73 - protein, 12 aa, synthetic construct
ARKDYYYGIMDV